SEQUENCE LISTING (11 sequences)

SEQ ID NO: 1 - protein, 90 aa, synthetic construct
  REGION 1..90: note = Description of Artificial Sequence: Synthetic polypeptide
DDDGKTVDGPSPLVLRAFINGVNNGRNGLGSIYVFASGNGGIYEDNCNFDGYANSVFTITIGGIDKHGKRPKYSEACSSQLAVTYAGGSA

SEQ ID NO: 2 - protein, 90 aa, synthetic construct
  REGION 1..90: note = Description of Artificial Sequence: Synthetic polypeptide
PDDGKTMEGPDILVLRAFINGVQNGRDGKGSIYVFASGNGGGFEDNCNFDGYTNSIYSITVGAIDRKGLHPSYSEACSAQLVVTYSSGSG

SEQ ID NO: 3 - protein, 90 aa, Pneumocystis carinii
DDDGKTVDGPSPLVLRAFINGVNNGRNGLGSIYVFASGNGGIYDDNCNFDGYANSVFTITIGGIDKHGKRFAYSEACSSQLAVTYAGGSA

SEQ ID NO: 4 - DNA, 270 bp, Pneumocystis carinii
  source 1..270: mol_type = other DNA
gatgacgatggaaaaaccgttgatgggccttctcctcttgttcttagagcatttattaatggagtaaataatgggaggaatgggttgggttctatctatgtttttgcatcaggaaatggcggaatatacgatgacaactgtaattttgatggatatgcaaatagcgtgtttactattactattggtggtatagataaacacggaaagcgctttgcatattctgaagcgtgttcttctcagttagctgttacatatgcaggcggaagtgca

SEQ ID NO: 5 - protein, 844 aa, Aspergillus fumigatus
MRFLGSIALVLSSISVASANVRSRSYDTHEFFALHLDDSASPSHVAQLLGARHEGQIGELANHHTFSIPRERSSDLDALLERARAARKIRRRARDDATSQEQHNDALGGILWSQKLAPKKRLVKRVPPPERLARTFATGKEDPVAAQSQKRIASTLGITDPIFNGQWHLFNTVQLGHDLNVTGVWMEGITGKGVTTAVVDDGLDMYSNDLKPNYFPEGSYDFNDHTPEPRPRLSDDKHGTRCAGEIAAARNDVCGVGVAYDSRVAGVRILSKAIDDADEATAINFAYQENDIFSCSWGPPDDGATMEGPGILIKRAFVNGVQNGRGGKGSIFVFAAGNGASFEDNCNFDGYTNSIYSITVGAIDREGNHPSYSESCSAQLVVAYSSGSGDAIHTTDVGTDKCYSFHGGTSAAGPLAAGTVALALSARPELTWRDAQYLMVETAVPIHEDDGSWQVTKAGRKFSHDWGYGKVDAYALVQKAKTWELVKPQAWFHSPWLRVQHKVPQGDQGLASSYEVTEQMMKNANIARLEHVTVTMNVNHTRRGDLSVELRSPEGIVSHLSTTRKSDNEKAGYVDWTFMTVAHWGESGVGRWTVIVKDTNVNEFTGEFIDWRLNLWGEAIDGANQKPHPFPDEHDDDHSIEDAIVATTSVETGPTKTGVPGSTDDTINRPVNAKPVETQTPSPAETTATKLAPPAETRPAATATSSPTPPAASDSFLPSFMPTFGASKRTQIWIYAAIGSIIVFCIGLGIYFQVQRRKRILNNPRDDYDFEMIEDENALHGGNGRSGRTQRRGGELYNAFAGESDEEEPLFSDEDDEPYRDRAPSEDRLRDTSSDDRSLRHGDH

SEQ ID NO: 6 - protein, 924 aa, Candida albicans
MLPIKLLIFILGYLLSPTLQQYQQIPPRDYENKNYFLVELNTTNSQKPLIDFISHYRGHYNFEHQLSSLDNHYVFSIDKSHPHNSFLGNHNSNEYNLMKRQLGHEQDYDELISHVESIHLLPMKKLSKRIPVPIEMEDVVFDNRDDTGSDNHEATDEAHQKLIEIAKKLDIHDPEFTTQWHLINLKYPGHDVNVTGLWLEDILGQGIVTALVDDGVDAESDDIKQNFNSEGSWDFNNKGKSPLPRLFDDYHGTRCAGEIAAVKNDVCGIGVAWKSQVSGIRILSGPITSSDEAEAMVYGLDTNDIYSCSWGPTDNGKVLSEPDVIVKKAMIKGIQEGRDKKGAIYVFASGNGGRFGDSCNFDGYTNSIYSITVGAIDYKGLHPQYSEACSAVMVVTYSSGSGEHIHTTDIKKKCSATHGGTSAAAPLASGIYSLILSANPNLTWRDVQYISVLSATPINEEDGNYQTTALNRKYSHKYGYGKTDAYKMVHFAKTWVNVKPQAWYYSDIIEVNQTITTTPEQKAPSKRDSPQKIIHSSVNVSEKDLKIMNVERVEHITVKVNIDSTYRGRVGMRIISPTGVISDLATFRVNDASTRGFQNWTFMSVAHWGETGIGEWKVEVFVDDSKGDQVEINFKDWQFRIFGESIDGDKAEVYDITKDYAAIRRELLEKEKQNSKSTTTTSSTTTATTTSGGEGDQKTTTSAENKESTTKVDNSASITTSQTASLTSSNEQHQPTESNSDSDSDTDDENKQEGEEDNDNDNDNGNKKANSDNTGFYLMSIAVVGFIAVLLVMKFHKTPGSGRRRRRRDGYEFDIIPGEDYSDSDDDEDDSDTRRADDDSFDLGHRNDQRVVSASQQQRQYDRQQDEARDRLFDDFNAESLPDYENDMFKIGDEEEEEEEEEEGQQSAKAPSNSEGNSGTSTKK

SEQ ID NO: 7 - protein, 917 aa, Cryptococcus neoformans
MRTLLSLWGILLALIVPPSLALQRPQPRSYDTHAYYALELDPSISPAAALQLSKSLGVELVERIGELDGHWLVRTEGWTPEHASITKRSVSHDPILKRWEALPSSLGKKSLTPLSLKQRAKRHKSYSPRSRHSRDDRTELLYAQNELHLADPMLDQQWHLINTQMKDIELNVTGLWGRGITGEGVHVVIIDDGLDVESKDLKDNFFAEGSYDFNDHTELPIPRLKDDQHGTRCAGEIAAVPNDVCGVGVAYDSKIAGVRILSAPISDADEAAALNYAYQLNDIYSCSWGPPDDGRSMEAPDGLILKAMVNGVQKGRDGKGSVFVFAAGNGGGSDDQCNFDGYTNSIFSVTVGAVDRKGLHPYYSEMCAAMMVVAPSSGSGDHIHTTDVGKDKCSHSHGGTSAAAPLAVGVFALALSVRPDLTWRDIQHLAVRHAVFFNPDDPAWELTAAGRHFSYKYGYGKLDAGLFVEAAEKWQLVKPQTWYDSPSVYLPTTSPADVTRRQDEAADGPTSSDEETSNPPPVVEPSGSFITEDGVISTYEVTQSMLFDANFERLEHVTVRVWIDHQRRGDVEVELTSPNGVVSVLCRQRRFDNADSGFPGWKFMSLKHWDENPVGTWTIKVKDQVNPDKTGRFVAWSLQLWGESVDPALAKLWAPAEEGQPDEEQTGSNPSTTVSQKPKPTALLPGDHGEASGEATQPGLGSATAHPQPTSTTGDAGNVAEPTGPTDADADEGFFSGISNLASSSTWLAGAGAIIILSGAAIGAFFFIRARRQKRNLFGLSNNGQGARGAYEPVDDVQMSLLERGRRKFGKSKSESQGTKDLYDAFGDGPSDEEEEDLDERTALRYHDGFLEDDEPNEVGPKTEYKDEPESEPETFKDGEETVGTKDKGKGKGPSEGESGSGSSSSWQDAADEEARV

SEQ ID NO: 8 - protein, 90 aa, Pneumocystis sp.
DDDGKTVDGPSSLVLRALINGVNNGRNGLGSIYVFASGNGGIYEDNCNFDGYANSVFTITIGGIDKHGKRLKYSEACSSQLAVTYAGGSA

SEQ ID NO: 9 - protein, 90 aa, Aspergillus fumigatus
PDDGATMEGPGILIKRAFVNGVQNGRGGKGSIFVFAAGNGASFEDNCNFDGYTNSIYSITVGAIDREGNHPSYSESCSAQLVVAYSSGSG

SEQ ID NO: 10 - protein, 90 aa, Cryptococcus neoformans
PDDGRSMEAPDGLILKAMVNGVQKGRDGKGSVFVFAAGNGGGSDDQCNFDGYTNSIFSVTVGAVDRKGLHPYYSEMCAAMMVVAPSSGSG

SEQ ID NO: 11 - protein, 90 aa, Candida albicans
TDNGKVLSEPDVIVKKAMIKGIQEGRDKKGAIYVFASGNGGRFGDSCNFDGYTNSIYSITVGAIDYKGLHPQYSEACSAVMVVTYSSGSG

(19) United States
(12) Patent Application Publication
(10) Pub. No.: US 2026/0232775 A1
(43) Pub. Date: Aug. 13, 2026

(54) TREATMENT AND DETECTION OF INFECTION AND DISEASE ASSOCIATED WITH DIFFERENT FUNGAL PATHOGENS

(71) Applicant: University of Georgia Research Foundation, Inc., Athens, GA (US)

(72) Inventors: Karen A. Norris, Athens, GA (US); Whitney Rabacal, Athens, GA (US); Emily Rayens, Athens, GA (US)

(73) Assignee: University of Georgia Research Foundation, Inc., Athens, GA (US)

(21) Appl. No.: 19/344,838

(22) Filed: Sep. 30, 2025

Related U.S. Application Data
(62) Division of application No. 17/425,279, filed on Jan. 23, 2020, now Pat. No. 12,453,761.
(60) Provisional application No. 62/796,120, filed on Jan. 24, 2019.

(51) Int. Cl. A61K 39/00; A61P 31/10; C07K 14/37; C07K 16/14

(52) U.S. Cl. CPC A61K 39/0002; A61P 31/10; C07K 14/37; C07K 16/14; A61K 2039/55511

(57) ABSTRACT

Described are immunogenic compositions comprising a non-naturally occurring pan-fungal Kex peptide, and methods of using such compositions for the treatment or prevention of infection and/or diseases associated with fungal pathogens (e.g., Pneumocystis, Aspergillus, Candida, or Cryptococcus) in the subject. Also provided are compositions and kits for detecting or quantifying the presence of antibodies directed against a non-naturally occurring pan-fungal Kex peptide in a subject.

### CROSS-REFERENCE TO RELATED APPLICATION

This application is a divisional of U.S. application Ser. No. 17/425,279, filed Jul. 22, 2021, which is a U.S. national phase application, pursuant to 35 U.S.C. § 371, of PCT International Application No. PCT/US2020/014805, filed Jan. 23, 2020, which claims priority to and benefit of U.S. Provisional Application No. 62/796,120, filed on Jan. 24, 2019, the entire contents of each of which are hereby incorporated by reference herein.

### SEQUENCE LISTING

This application contains a Sequence Listing which has been submitted electronically in XML format and is hereby incorporated by reference in its entirety. The Sequence Listing XML file, created on Apr. 15, 2026, is named 173093_010808USDIV_SL.xml, and is 14,958 bytes in size.

### BACKGROUND

Pathogenic fungal organisms are universal in the environment and can cause global health threats. Pathogenic fungi are typically not harmful to individuals with healthy and normally functional immune systems even after exposure, for example, by inhalation. However, individuals with weakened or compromised immune systems, or those having pre-existing medical conditions, such as those with lung diseases or viral infections, such as HIV/AIDS infection, are at a higher risk of developing serious health problems and adverse reactions following exposure to and infection by fungal pathogens.

Because of the grave repercussions of infection by fungal organisms in individuals in poor medical health and in those with weakened immune systems, there is an ongoing and urgent need for methods and compositions for treating or preventing infection and associated diseases caused by these pathogens. Cost-effective and efficient methods of treatment of and protection from fungal pathogens are required, particularly in less affluent parts of the world. Effective methods and approaches for treating or preventing infection by fungal pathogens for which suitable therapies are currently nonexistent or inadequate are particularly desirable to alleviate fungal pathogens that pose ever-present threats to at-risk individuals worldwide.

### SUMMARY

Methods for treating or preventing infection (e.g., opportunistic infection) by fungal pathogens, as well as treating or preventing pulmonary disease and poor pulmonary function associated with infection by fungal pathogens are provided and described herein.

In an aspect, an immunogenic peptide or a polynucleotide encoding an immunogenic peptide having at least about 95% or greater amino acid sequence identity to

(Pan-fungal peptide 2)
SEQ ID NO: 2
PDDGKTMEGPDILVLRAFINGVQNGRDGKGSIYVFASGNGGGFEDNCNFD
GYTNSIYSITVGAIDRKGLHPSYSEACSAQLVVTYSSGSG;
or
(Pan-fungal peptide 1)
SEQ ID NO: 1
DDDGKTVDGPSPLVLRAFINGVNNGRNGLGSIYVFASGNGGIYEDNCNFD
GYANSVFTITIGGIDKHGKRPKYSEACSSQLAVTYAGGSA,

In another aspect is provided an immunogenic peptide or a polynucleotide encoding an immunogenic peptide comprising or consisting of

(Pan-fungal peptide 2)
SEQ ID NO: 2
PDDGKTMEGPDILVLRAFINGVQNGRDGKGSIYVFASGNGGGFEDNCNFD
GYTNSIYSITVGAIDRKGLHPSYSEACSAQLVVTYSSGSG;
or
(Pan-fungal peptide 1)
SEQ ID NO: 1
DDDGKTVDGPSPLVLRAFINGVNNGRNGLGSIYVFASGNGGIYEDNCNFD
GYANSVFTITIGGIDKHGKRPKYSEACSSQLAVTYAGGSA,

In some embodiments, the immunogenic composition comprises an effective amount of an immunogenic peptide or a polynucleotide encoding an immunogenic peptide described supra and a pharmaceutically acceptable excipient. In some embodiments, the immunogenic composition further comprises an adjuvant. In an embodiment, the adjuvant is alpha-galactosylceramide (αGC). In an embodiment, the adjuvant is alum.

In another aspect, a method of eliciting an immune response in a subject is provided, in which the method comprises administering to the subject a peptide, polynucleotide, or immunogenic composition as described supra.

In another aspect, a method is provided herein for treating or protecting a subject against a fungal infection, the method comprising administering to the subject an immunogenic peptide, polynucleotide, or composition described supra.

Also provided herein are methods of treating or protecting a subject against a fungal infection, the methods comprising administering to the subject an immunogenic peptide, polynucleotide, or composition of any previous claim, an isolated antiserum comprising an antibody that specifically binds an immunogenic peptide of any previous claim or an antigen-binding fragment thereof, or an isolated or purified antibody or an antigen-binding fragment that specifically binds an immunogenic peptide of any previous claim in an amount effective to treat or protect a subject against a fungal infection. In some embodiments, the antibody is a monoclonal antibody, polyclonal antibody, or an antigen-binding fragment thereof.

In some embodiments of the methods disclosed herein, the fungal infection is associated with Aspergillus, Candida, and/or Cryptococcus. In some embodiments, the methods treat pulmonary disease, pulmonary dysfunction, or a symptom thereof. In some embodiments, the pulmonary disease or pulmonary dysfunction is Pneumocystis pneumonia. In some embodiments, the pulmonary disease or pulmonary dysfunction is aspergillosis.

In another aspect, method of treating or protecting a subject against fungal infection is provided, in which the method involves administering to the subject an immunogenic composition comprising SEQ ID NO: 2 or fragment thereof or a polynucleotide encoding SEQ ID NO: 2 or a fragment thereof in an amount effective to induce an immune response against one or more of Pneumocystis, Aspergillus, Candida, or Cryptococcus fungal pathogens.

In another aspect, a method of treating or protecting a subject against fungal infection is provided, in which the method comprises administering to the subject an effective amount of an antibody or an antigen-binding fragment thereof that specifically binds SEQ ID NO: 2. In some embodiments, the antibody or fragment thereof is present in or isolated from an antiserum derived from a donor subject. In some embodiments, the antibody or an antigen-binding fragment thereof specifically binds a Kex peptide of Pneumocystis, Aspergillus, Candida, or Cryptococcus.

In another aspect, a method is provided for eliciting an immune response in a subject, the method comprising administering to the subject a wildtype Kex peptide, or a polynucleotide encoding the same, and alpha-galactosylceramide (α-GC).

In yet another aspect, a method of treating or protecting a subject against a fungal infection is provided, the method comprising administering to the subject a wildtype Kex peptide, or a polynucleotide encoding the same, and alpha-galactosylceramide (α-GC). In some embodiments of this method, the fungal infection is associated with Aspergillus, Candida, and/or Cryptococcus. In some embodiments of this method or the method for eliciting an immune response in a subject comprising administering to the subject a wildtype Kex peptide or a polynucleotide encoding the same and α-GC, the method treats pulmonary disease, pulmonary dysfunction, or a symptom thereof.

Another aspect provides a method of treating or protecting a subject against fungal infection, in which the method comprises administering to the subject an immunogenic composition comprising a wildtype Kex peptide or a fragment thereof, or a polynucleotide encoding the same, and alpha-galactosylceramide (α-GC) in an amount effective to induce an immune response against one or more of Pneumocystis, Aspergillus, Candida, or Cryptococcus fungal pathogens.

In yet another aspect, a method of treating or protecting a subject against infection by Aspergillus or Pneumocystis fungal pathogens is provided, in which the method involves administering to the subject an immunogenic composition comprising a Kex peptide of SEQ ID NO: 2 (Pan-fungal peptide 2), or a fragment thereof, or a polynucleotide encoding the Pan-fungal peptide 2 peptide or the fragment thereof, in an amount effective to induce an immune response against Pneumocystis and Aspergillus fungal pathogens. In an embodiment of the method, the subject is also administered an adjuvant. In an embodiment, the adjuvant is alpha-galactosylceramide (α-GC). In another embodiment, the adjuvant is alum. In embodiments of the method, the induced immune response treats or protects the subject against pulmonary disease or pulmonary dysfunction and/or the symptoms thereof, or against Aspergillus-associated disease and/or the symptoms thereof. In an embodiment of the method, the subject has an immune deficiency disease or am acquired immune deficiency disease syndrome, e.g., HIV, AIDS, or SIV. In certain embodiments the pulmonary disease is Pneumocystis pneumonia (PCP) and/or the symptoms thereof, and the Aspergillus-associated disease is aspergillosis or Invasive Pulmonary Aspergillosis (IPA) and/or the symptoms thereof.

In some embodiments of any of the methods disclosed herein, the subject is a mammal, and in some embodiments, the subject is a human. In some embodiments, the subject is immunocompromised or immunosuppressed. In some embodiments, the immunocompromised or immunosuppressed subject is being treated for cancer, an immunodeficiency disease, a congenital disease, or an autoimmune disease. The immunocompromised or immunosuppressed subject in some embodiments is a pre-transplant subject or a post-transplant subject. In some embodiments, the immunocompromised or immunosuppressed subject is being treated for HIV, rheumatoid arthritis, or psoriasis.

Other features and advantages of the described embodiments will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in the embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Kex1” or “Kexin” or “KEX” protein is meant a polypeptide or peptide fragment thereof having at least about 85% or greater amino acid identity to the amino acid sequence provided at GenBank Accession No. EU918304.1, at NCBI Accession No. XM_746441.1, at GenBank Accession No. AF022372.1, or at NCBI Accession No. XM_572303.1 and having immunogenic activity. In an embodiment, the Kex peptide is an antigenically stable active site peptide sequence. (Kutty, G. and Kovacs, J. A., 2003, Infect. Immun., 71(1):571-574; Lee. L. H. et al., 2000, Gene, 242(1-2):141-150; and Russian, D. A. et al., 1999, Proc. Assoc. Am. Physicians, 111(4):347-356). In one embodiment, a Kex peptide is a fragment of a naturally occurring Kexin protein or is a non-naturally occurring pan-fungal peptide or fragment thereof.

By “Pan-fungal peptide 1” is meant a KEX peptide comprising a sequence having 95, 96, 97, 98, 99, or 100% identity to the amino acid sequence of Pan-fungal peptide 1 and having immunogenic activity. In some embodiments, a Pan-fungal peptide 1 comprises 1, 2, or 3 additional amino acids at the carboxy and/or amino terminus of the peptide, which additional amino acids do not alter the peptide's ability to stimulate an immune response in a subject. The sequence of Pan-fungal peptide 1 follows:

(Pan-fungal peptide 1)
(SEQ ID NO: 1)
DDDGKTVDGPSPLVLRAFINGVNNGRNGLGSIYVFASGNGGIYEDNCNFD
GYANSVFTITIGGIDKHGKRPKYSEACSSQLAVTYAGGSA,

By “Pan-fungal peptide 2” is meant a KEX peptide comprising a sequence having 95, 96, 97, 98, or 99% identity to Pan-fungal peptide 2 and having immunogenic activity. In some embodiments, a Pan-fungal peptide 2 comprises 1, 2, or 3 additional amino acids at the carboxy and/or amino terminus, which do not change the peptide's immunogenicity. The sequence of Pan-fungal peptide 2 follows:

(Pan-fungal peptide 2)
(SEQ ID NO: 2)
PDDGKTMEGPDILVLRAFINGVQNGRDGKGSIYVFASGNGGGFEDNCNFD
GYTNSIYSITVGAIDRKGLHPSYSEACSAQLVVTYSSGSG,.

An exemplary Kex1 polypeptide fragment of Pneumocystis isolated from Pneumocystis colonized non-human primates (cynomolgus macaques) and having GenBank Accession No. EU918304.1 is provided below:

| (SEQ ID NO: 3) | |
|---|---|
| 1 | DDDGKTVDGP SPLVLRAFIN GVNNGRNGLG SIYVFASGNG GIYDDNCNFD GYANSVFTIT |
| 61 | IGGIDKHGKR FAYSEACSSQ LAVTYAGGSA |

An exemplary polynucleotide sequence encoding the Kex1 polypeptide fragment provided at GenBank Accession No. EU918304.1 is provided below:

| (SEQ ID NO: 4) | |
|---|---|
| 1 | gatgacgatg gaaaaaccgt tgatgggcct tctcctcttg ttcttagagc atttattaat |
| 61 | ggagtaaata atgggaggaa tgggttgggt tctatctatg tttttgcatc aggaaatggc |
| 121 | ggaatatacg atgacaactg taattttgat ggatatgcaa atagcgtgtt tactattact |
| 181 | attggtggta tagataaaca cggaaagcgc tttgcatatt ctgaagcgtg ttcttctcag |
| 241 | ttagctgtta catatgcagg cggaagtgca |

An exemplary Kex (KexB endoprotease) polypeptide sequence of Aspergillus fumigatus (Af293) having NCBI Accession No. XM_746441.1 is provided below:

(SEQ ID NO: 5)
MRFLGSIALVLSSISVASANVRSRSYDTHEFFALHLDDSASPSHVAQLLG
ARHEGQIGELANHHTFSIPRERSSDLDALLERARAARKIRRRARDDATSQ
EQHNDALGGILWSQKLAPKKRLVKRVPPPERLARTFATGKEDPVAAQSQK
RIASTLGITDPIFNGQWHLFNTVQLGHDLNVTGVWMEGITGKGVTTAVVD
DGLDMYSNDLKPNYFPEGSYDFNDHTPEPRPRLSDDKHGTRCAGEIAAAR
NDVCGVGVAYDSRVAGVRILSKAIDDADEATAINFAYQENDIFSCSWGPP
DDGATMEGPGILIKRAFVNGVQNGRGGKGSIFVFAAGNGASFEDNCNFDG
YTNSIYSITVGAIDREGNHPSYSESCSAQLVVAYSSGSGDAIHTTDVGTD
KCYSFHGGTSAAGPLAAGTVALALSARPELTWRDAQYLMVETAVPIHEDD
GSWQVTKAGRKFSHDWGYGKVDAYALVQKAKTWELVKPQAWFHSPWLRVQ
HKVPQGDQGLASSYEVTEQMMKNANIARLEHVTVTMNVNHTRRGDLSVEL
RSPEGIVSHLSTTRKSDNEKAGYVDWTFMTVAHWGESGVGRWTVIVKDTN
VNEFTGEFIDWRLNLWGEAIDGANQKPHPFPDEHDDDHSIEDAIVATTSV
ETGPTKTGVPGSTDDTINRPVNAKPVETQTPSPAETTATKLAPPAETRPA
ATATSSPTPPAASDSFLPSFMPTFGASKRTQIWIYAAIGSIIVFCIGLGI
YFQVQRRKRILNNPRDDYDFEMIEDENALHGGNGRSGRTQRRGGELYNAF
AGESDEEEPLFSDEDDEPYRDRAPSEDRLRDTSSDDRSLRHGDH

An exemplary Kex (Kex2 proteinase) polypeptide sequence of Candida albicans having GenBank Accession No. AF022372.1 is provided below:

(SEQ ID NO: 6)
MLPIKLLIFILGYLLSPTLQQYQQIPPRDYENKNYFLVELNTTNSQKPLI
DFISHYRGHYNFEHQLSSLDNHYVFSIDKSHPHNSFLGNHNSNEYNLMKR
QLGHEQDYDELISHVESIHLLPMKKLSKRIPVPIEMEDVVFDNRDDTGSD
NHEATDEAHQKLIEIAKKLDIHDPEFTTQWHLINLKYPGHDVNVTGLWLE
DILGQGIVTALVDDGVDAESDDIKQNFNSEGSWDFNNKGKSPLPRLFDDY
HGTRCAGEIAAVKNDVCGIGVAWKSQVSGIRILSGPITSSDEAEAMVYGL
DTNDIYSCSWGPTDNGKVLSEPDVIVKKAMIKGIQEGRDKKGAIYVFASG
NGGRFGDSCNFDGYTNSIYSITVGAIDYKGLHPQYSEACSAVMVVTYSSG
SGEHIHTTDIKKKCSATHGGTSAAAPLASGIYSLILSANPNLTWRDVQYI
SVLSATPINEEDGNYQTTALNRKYSHKYGYGKTDAYKMVHFAKTWVNVKP
QAWYYSDIIEVNQTITTTPEQKAPSKRDSPQKIIHSSVNVSEKDLKIMNV
ERVEHITVKVNIDSTYRGRVGMRIISPTGVISDLATFRVNDASTRGFQNW
TFMSVAHWGETGIGEWKVEVFVDDSKGDQVEINFKDWQFRIFGESIDGDK
AEVYDITKDYAAIRRELLEKEKQNSKSTTTTSSTTTATTTSGGEGDQKTT
TSAENKESTTKVDNSASITTSQTASLTSSNEQHQPTESNSDSDSDTDDEN
KQEGEEDNDNDNDNGNKKANSDNTGFYLMSIAVVGFIAVLLVMKFHKTPG
SGRRRRRRDGYEFDIIPGEDYSDSDDDEDDSDTRRADDDSFDLGHRNDQR
VVSASQQQRQYDRQQDEARDRLFDDFNAESLPDYENDMFKIGDEEEEEEE
EEEGQQSAKAPSNSEGNSGTSTKK

An exemplary Kex polypeptide sequence of Cryptococcus neoformans (JEC21) having NCBI Accession No. XM_572303.1 is provided below:

(SEQ ID NO: 7)
MRTLLSLWGILLALIVPPSLALQRPQPRSYDTHAYYALELDPSISPAAAL
QLSKSLGVELVERIGELDGHWLVRTEGWTPEHASITKRSVSHDPILKRWE
ALPSSLGKKSLTPLSLKQRAKRHKSYSPRSRHSRDDRTELLYAQNELHLA
DPMLDQQWHLINTQMKDIELNVTGLWGRGITGEGVHVVIIDDGLDVESKD
LKDNFFAEGSYDFNDHTELPIPRLKDDQHGTRCAGEIAAVPNDVCGVGVA
YDSKIAGVRILSAPISDADEAAALNYAYQLNDIYSCSWGPPDDGRSMEAP
DGLILKAMVNGVQKGRDGKGSVFVFAAGNGGGSDDQCNFDGYTNSIFSVT
VGAVDRKGLHPYYSEMCAAMMVVAPSSGSGDHIHTTDVGKDKCSHSHGGT
SAAAPLAVGVFALALSVRPDLTWRDIQHLAVRHAVFFNPDDPAWELTAAG
RHFSYKYGYGKLDAGLFVEAAEKWQLVKPQTWYDSPSVYLPTTSPADVTR
RQDEAADGPTSSDEETSNPPPVVEPSGSFITEDGVISTYEVTQSMLFDAN
FERLEHVTVRVWIDHQRRGDVEVELTSPNGVVSVLCRQRRFDNADSGFPG
WKFMSLKHWDENPVGTWTIKVKDQVNPDKTGRFVAWSLQLWGESVDPALA
KLWAPAEEGQPDEEQTGSNPSTTVSQKPKPTALLPGDHGEASGEATQPGL
GSATAHPQPTSTTGDAGNVAEPTGPTDADADEGFFSGISNLASSSTWLAG
AGAIIILSGAAIGAFFFIRARRQKRNLFGLSNNGQGARGAYEPVDDVQMS
LLERGRRKFGKSKSESQGTKDLYDAFGDGPSDEEEEDLDERTALRYHDGF
LEDDEPNEVGPKTEYKDEPESEPETFKDGEETVGTKDKGKGKGPSEGESG
SGSSSSWQDAADEEARV

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies of the described embodiments may exist in a variety of forms including, for example, polyclonal antibodies, monoclonal antibodies, Fv, Fab and F(ab′)2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods, 231:25-38), composed of either a V_{L} or a V_{H} domain which exhibit sufficient affinity for the target, and multispecific antibodies formed from antibody fragments. The antibody fragment also includes a human antibody or a humanized antibody or a portion of a human antibody or a humanized antibody.

Antibodies can be made by any of the methods known in the art utilizing a polypeptide (e.g., a Kexin polypeptide), or immunogenic peptide fragments thereof, as an immunogen. One method of obtaining antibodies is to immunize suitable host animals with an immunogen and to follow standard procedures for polyclonal or monoclonal antibody production. The immunogen facilitates the presentation of the immunogenic fragments on the cell surface. Immunization of a suitable host can be carried out in a number of ways. Nucleic acid sequences encoding a polypeptide as described herein, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. To produce large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

By “anti-Kexin antibody,” “anti-Kex antibody,” or “anti-Kex1 antibody” is meant an antibody or an antigen binding fragment thereof that selectively binds a Kexin polypeptide or a peptide fragment thereof, including, for example, a Kex1 peptide fragment of a fungal pathogen, such as Candida albicans, Pneumocystis hominis, Pneumocystis jirovecii (aka carinii), Aspergillus fumigatus and Cryptococcus neoformans as described herein. In various embodiments, anti-Kexin antibody or anti-Kex1 antibody specifically binds a binding site of a Kexin protein or peptide. In specific embodiments, the anti-Kexin antibody or anti-Kex1 antibody specifically binds a binding site of a Kexin protein or peptide of a fungal pathogen, for example, one or more of a Candida, Pneumocystis, Aspergillus and/or Cryptococcus fungal pathogen.

An “antiserum” refers to blood serum that contains one or more antibodies directed against a specific antigen. Antiserum containing antibodies may be obtained from the blood or serum of an animal (a mammal), including a human, that has been immunized or inoculated with an immunogen (or an antigen material) either by injection, typically into the bloodstream or tissues, or by infection. In an embodiment, the animal (a mammal), including a human, may be immunized or inoculated with the blood or serum of an organism or individual whose immune system has been stimulated to generate an immune response (e.g., antibody production) by infection or natural contact with an antigenic material or immunogen. In this case, an antiserum contains anti-Kex peptide antibodies, e.g., polyclonal antibodies or populations of monoclonal antibodies, generated or produced by an immunized, inoculated, or exposed donor subject against a Kex peptide immunogen, or a polynucleotide encoding the Kex peptide immunogen, derived from a fungal pathogen, e.g., Pneumocystis (e.g., Pneumocystis jirovecii). Such antiserum, isolated (and/or purified) from the donor subject is used to immunize (i.e., administer to) another (unrelated) subject to provide immunity (acquired immunity) against infection or disease caused by or associated not only with the Pneumocystis pathogen as original source of the immunogen, but also with other fungal pathogens that have a Kex peptide that is also targeted and recognized by the antibodies in the antiserum. In embodiments, the fungal pathogens include Pneumocystis species (spp.) and one or more of Candida spp. or Candida albicans, Aspergillus spp. or Aspergillus fumigatus, or Cryptococcus spp. or Cryptococcus neoformans. In this way, a subject who receives the antiserum, i.e., antibodies in the antiserum, is treated or protected against infection and/or disease caused by more than one fungal pathogen. Such antiserum-derived immunoprotection against multiple fungal pathogens constitutes an acquired or passive immunity obtained by the recipient subject and imparted from the donor subject's isolated antiserum. As will be appreciated by one skilled in the art, blood serum is the amber-colored, protein-rich liquid component of blood that separates from the clot when blood coagulates. The serum component containing one or more antibodies (cross-protective antibodies) is termed “antiserum.” In an embodiment, the antiserum is an isolated antiserum, e.g., isolated from a donor subject. In an embodiment, an isolated antiserum may be processed by methods used by one skilled in the art, such as dilution, concentration (e.g., via filtration or centrifugation or both), chromatography, purification to remove ions or extraneous protein, and the like, prior to its use as a treatment or protective therapeutic as described herein. In an embodiment, an isolated antiserum may be further purified after isolation. In an embodiment, an isolated antiserum is not further processed or purified. In an embodiment, antibodies, or antigen-binding fragments thereof, contained in an isolated antiserum may be further isolated by methods practiced by those having skill in the art, such as, without limitation, by affinity chromatography, size exclusion chromatography, immunoprecipitation, dialysis, HPLC chromatography, etc.

By “biological sample” is meant any liquid, cell, or tissue obtained from a subject. In some embodiments, the biological sample is blood, serum, plasma, cerebrospinal fluid, bronchoalveolar lavage, sputum, tears, saliva, urine, semen, feces, etc.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte that is detected or that is to be detected.

By “disease” is meant any condition, dysfunction, or disorder that damages or interferes with the normal function of a cell, tissue, or organ. In various embodiments, the disease is a pulmonary (lung) disease or a brain disease, e.g., meningitis. Non-limiting examples of pulmonary diseases include Chronic Obstructive Pulmonary Disease (COPD), which is a progressive lung disease that includes emphysema, chronic bronchitis, refractory (non-reversible) asthma, pneumonia (e.g., Pneumocystis pneumonia) and some forms of bronchiectasis.

By “effective amount” is meant the amount of an agent, e.g., active compound, molecule, protein, nucleic acid, etc., required to ameliorate the symptoms of a disease relative to an untreated patient. The effective amount of active compound(s) used to practice the methods as described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. An immunologically effective amount of an isolated antiserum as described herein is an amount required to treat a fungal infection or disease associated with one or more of the fungal pathogens described herein. By way of example, an effective amount of an isolated antiserum may be determined by measuring the amount or titer of antibodies directed against the desired immunogen present in the serum by methods known and practiced in the art. The range of typical dosages for passive immunotherapy (i.e., the administration of antiserum containing antibodies) includes about 0.3 mg to about 100 mg/kg of total body weight. Following passive immunotherapy, treatment efficacy is typically conducted, as individual patients respond differently to therapies. Adjustment of the dosage may be modified as needed. Treatment regimens can be determined by methods known and practiced by those having skill in the art. In one embodiment, the amount is sufficient to induce an immune response.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “genetic vaccine” is meant an immunogenic composition comprising a polynucleotide encoding an antigen.

By “immune response” is meant is any activity of the immune system that is generated against an antigen. In some embodiments, the immune response is an innate or an adaptive immune response that protects a subject from infection with a pathogen (e.g., fungal pathogen) or treats a pathogen infection. In some embodiments, an immune response involves the generation of antibodies against an antigen.

The term “immunocompromised” refers to a subject having a weakened or impaired immune system and/or associated immune response to a pathogen, pathogenic antigen, disease, etc. A subject may be immunocompromised as a consequence of taking immunosuppressive drugs, or by being afflicted with a disease or pathology that affects the subject's immune system, such as certain congenital diseases. The term “immunosuppressed” refers to a subject whose immune system and associated immune response to pathogens, pathogenic antigens, disease, etc. is partially or completely suppressed, for example, by a reduction in the activity or efficiency in the immune system. Immunosuppression of a subject's immune system or immune response may occur naturally due to a disease or disorder in the subject, or may be induced in the subject by the administration of immunosuppressive agents, drugs, e.g., anti-cancer drugs, compounds, and the like. In some cases, a subject who is immunosuppressed or is undergoing immunosuppression, or who has a weakened immune system due to a disease or condition (e.g., chemotherapy or an immune deficiency disease) is said to be immunocompromised.

By “immunogenic composition” is meant a composition comprising an antigen or immunogen or a polynucleotide encoding the antigen or immunogen, wherein the composition elicits an immune response in an immunized subject.

The terms “isolated,” “purified,” or “biologically pure” refer to material that is free to varying degrees from components which normally accompany it as found in its native state or environment. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” or “isolated peptide” is meant a polypeptide or peptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide or peptide as described herein. An isolated polypeptide or peptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “opportunistic infection” is meant an infection caused by pathogens such as fungal pathogens, bacteria, viruses, protozoa, or parasites that take advantage of an opportunity to infect a subject (host) that is not normally available, for example, a host having a weakened immune system, an immunocompromised host, an immunosuppressed host, a host with altered microbiota or microflora, or a host having protective integumentary barriers that have been damaged or breached. In an embodiment, an opportunistic infection is caused by one or more fungal pathogens as described herein.

By “reduces” or “diminishes” is meant a negative alteration of at least 10%, 25%, 50%, 75%, or 100%.

By “reference” is meant a standard or control condition. A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody or antigen binding fragment thereof that recognizes and binds a polypeptide or peptide, but which does not substantially recognize and bind other molecules in a sample, for example, a biological sample, which naturally includes a polypeptide or peptide as described herein. Cross-reactive binding includes specific binding (e.g., by an antibody or an antigen binding fragment thereof) to an original polypeptide or peptide antigen/immunogen as well as binding to a polypeptide or peptide other than the original antigen/immunogen.

Nucleic acid molecules useful in generating a recombinant immunogen or a vaccine include any nucleic acid molecule that encodes a polypeptide or a peptide fragment thereof, such as a Kex wildtype or pan-fungal polypeptide or Kex wildtype or pan-fungal peptide described herein. Such nucleic acid molecules need not be 100% identical with an endogenous nucleic acid sequence, but will typically exhibit substantial identity to an endogenous sequence. Polynucleotides having “substantial identity” to an endogenous sequence are typically capable of hybridizing with at least one strand of a double-stranded nucleic acid molecule. Nucleic acid molecules may include any nucleic acid molecule that encodes a polypeptide or a peptide fragment thereof. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, or a murine, bovine, equine, canine, ovine, or feline mammal. In an embodiment, the subject is a human. In an embodiment, a subject is a human patient who is undergoing treatment for infection or disease caused by one or more pathogenic fungi, such as Pneumocystis, Aspergillus, Candida, or Cryptococcus. In an embodiment as subject is a human patient who is at risk of infection (e.g., opportunistic infection) or disease caused by one or more pathogenic fungi, such as Pneumocystis, Aspergillus, Candida, or Cryptococcus. In an embodiment, a subject is a mammalian (e.g., a human; a non-human primate) donor subject from whom antiserum containing anti-fungal Kex peptide antibodies is obtained or isolated. In an embodiment, a subject is a mammalian (e.g., a human; a non-human primate) recipient subject who receives an isolated antiserum and acquires protective immunity (and treatment) against multiple fungal pathogens.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. One skilled in the art will recognize that depending on the expression vector used, additional nucleotides may need to be added to the 5′ end of the foreign nucleic acid to be inserted into the vector to maintain the proper reading frame.

By “vaccine” is meant a preparation of immunogenic material (e.g., protein or nucleic acid; vaccine) capable of stimulating (eliciting) an immune response, administered to a subject to treat a disease, condition, or pathology, or to prevent a disease, condition, or pathology, such as an infectious disease (caused by Dengue virus infection, for example). The immunogenic material may include, for example, attenuated or killed microorganisms (such as attenuated viruses), or antigenic proteins, peptides or DNA derived from such microorganisms. Vaccines may elicit a prophylactic (preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous or intramuscular. Vaccines may also be administered with an adjuvant to boost the immune response.

Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, abating, diminishing, or ameliorating a disease, disorder and/or symptoms associated therewith. It will be appreciated that, although not precluded, treating a disease, disorder and/or symptoms associated therewith does not require that the disease, disorder, condition or symptoms associated therewith be eliminated.

As used herein, a therapeutic that “prevents” a disorder or condition refers to a compound or material that, in a statistical sample, reduces the occurrence of the disorder or condition in the treated sample relative to an untreated control sample, or delays the onset or reduces the severity of one or more symptoms of the disorder or condition relative to an untreated control sample. In an embodiment, a preventive therapeutic is an antibody or an antigen binding fragment thereof. In a particular embodiment, a preventive therapeutic is an isolated antiserum containing anti-Kex peptide antibodies or antigen binding fragments thereof as described herein.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

Exemplary embodiments as described herein are provided below as drawings and figures related to the described embodiments in their various and nonlimiting aspects.

FIG. 1 presents a multisequence alignment comparing the amino acid sequences of two pan-fungal Kex peptides, Pan-fungal peptide 1 (SEQ ID NO: 1) and Pan-fungal peptide 2 (SEQ ID NO: 2) derived from Pneumocystis (Human and Macaque) with the amino acid sequences of human Pneumocystis Kex1 peptide (SEQ ID NO: 8), macaque Pneumocystis Kex1 peptide (SEQ ID NO: 3), Aspergillus Kex peptide (SEQ ID NO: 9), Cryptococcus Kex peptide (SEQ ID NO: 10), and Candida Kex peptide (SEQ ID NO: 11).

FIG. 2 presents a multisequence alignment comparing the amino acid sequences of Pan-fungal peptide 1 (SEQ ID NO: 1) and Pan-fungal peptide 2 (SEQ ID NO: 2) with the amino acid sequence of human Pneumocystis Kex1 peptide (SEQ ID NO: 8). Pan-fungal peptide 1 shows 97% amino acid sequence identity to the human Pneumocystis Kex1 peptide, and Pan-fungal peptide 2 shows 69% amino acid sequence identity to the human Pneumocystis Kex1 peptide. As described herein (e.g., Example 1), Pan-fungal peptide 1 and Pan-fungal peptide 2 comprise the amino acid sequences shown in FIGS. 1 and 2, respectively, and as shown supra.

FIGS. 3A-3F illustrate pan-fungal peptide 2 prophylactic vaccination in SIV-infected Rhesus macaque. FIG. 3A is a timeline describing the administration and challenge schedule used to determine the immunogenicity of the pan-fungal 2 peptide in macaques. FIGS. 3B-3E graphically illustrate reciprocal endpoint titers (RETs) obtained by ELISA analysis of sera samples collected from a macaque monkey that had been immunized with Pan-fungal peptide 2 and alum adjuvant prior to SIV infection and challenge with Pneumocystis fungal pathogen, to determine serum immunoglobulin (IgG) titer against Cryptococcus Kex, Pneumocystis Kex1, and Aspergillus Kex as targets. For these figures, the arrows indicate when the test subject was immunized with Pan-fungal peptide 2 and when the test subject received the boost. The dotted vertical line shows when the test subject was infected with simian immunodeficiency virus (SIV). FIG. 3B is a graph of the RET measured via ELISA analysis of the Pan-fungal peptide 2. FIG. 3C is a graph of the RET measured via ELISA analysis for Cryptococcus Kex. FIG. 3D is a graph of the RET measured via ELISA analysis for Pneumocystis Kex1. FIG. 3E is a graph of the RET measured via ELISA analysis for Aspergillus Kex. FIG. 3F is a graph showing the percentage of Pneumocystis pneumonia (PcP) observed in animals vaccinated with pan-fungal 2 peptide (n=1) or a sham vaccine (n=7) up to 40 weeks after SIV infection.

FIGS. 4A-4C summarize prophylactic vaccination of SIV-infected Rhesus macaques with Pneumocystis Kex1 peptide and alum adjuvant followed by therapeutic vaccination of the animals with Pneumocystis Kex1 peptide and alpha-galactosylceramide (αGC) as adjuvant. FIG. 4A is a timeline describing the administration and challenge schedule used to study the effectiveness of Pneumocystis Kex1 prophylactic vaccination with alum as adjuvant and therapeutic vaccination with αGC as adjuvant. FIG. 4B is a graph of the RET measured via ELISA analysis of serum immunoglobulin G (IgG) titer against the target Pneumocystis Kex1. FIG. 4C is a graph showing the percentage of Pneumocystis pneumonia (PcP) observed in animals vaccinated with Pneumocystis Kex1 and alum adjuvant and boosted with Pneumocystis Kex1 with αGC adjuvant (n=6) or a sham vaccine (n=7) after SIV infection.

FIGS. 5A and 5B summarize Pneumocystis Kex1 therapeutic vaccination with αGC adjuvant in SIV-infected Rhesus macaques. FIG. 5A is a timeline describing the administration and challenge schedule used to study the immunogenicity of Pneumocystis Kex1 therapeutic vaccination with αGC. FIG. 5B is a graph of the RET measured via ELISA analysis serum immunoglobulin G (IgG) titer against the target Pneumocystis Kex1 during the late chronic phase of SIV infection.

FIGS. 6A and 6B illustrate Pan-fungal peptide 2-specific humoral responses following Pan-fungal peptide 2 immunization in mice. FIG. 6A depicts the immunization (vaccination) study design. CF-1 mice (7/group) were immunized with Pan-fungal peptide 2+adjuvant (TiterMax) or PBS+TiterMax (sham vaccination). Blood was collected for serologic analyses at 0, 14, and 28 days post-immunization. FIG. 6B is a graph illustrating the mean plasma reciprocal endpoint titer (RET) of Pan-fungal peptide 2-specific immunoglobulin G (IgG) titer, as determined by enzyme-linked immunosorbent assay (ELISA) of a single vaccination with Pan-fungal peptide 2+TiterMax1 or PBS+TiterMax. Pan-fungal peptide 2-vaccinated animals achieved a mean peak RET of 5×10⁵ compared with that of sham-vaccinated animals (**p 0.002).

FIG. 7 is a diagram illustrating the design of a study in which mice were vaccinated with Pan-fungal peptide 2 peptide and then challenged with Aspergillus fumigatus pathogen. Following the Pan-fungal peptide 2 peptide and sham vaccination regimens as described in FIG. 6A, all mice were immunosuppressed by administering 2.5 mg cortisone acetate per day for six days before intranasal challenge of 5×10⁶ Aspergillus (Af293) conidia spores. All mice were then observed twice daily for signs of invasive pulmonary aspergillosis (IPA). The association between Pan-fungal peptide 2 vaccination and IPA-related mortality is presented in Table 2 of Example 9, infra.

FIG. 8 is a graph showing lung fungal burden following A. fumigatus challenge, as described above in FIG. 7. Quantification of the fungal burden in the lungs was carried out using Grocott's methenamine silver (GMS) staining. A. fumigatus fungal burden was significantly reduced in Pan-fungal peptide 2-vaccinated animals (*p=0.026).

FIGS. 9A and 9B depict Pan-fungal peptide 2-specific humoral responses following Pan-fungal peptide 2immunization in non-human primates. FIG. 9A is a diagram depicting the vaccination study design. In this study, fifteen (15) Rhesus macaques (n=7 Pan-fungal peptide 2-vaccinated, n=8 sham-vaccinated) were immunized and boosted with Pan-fungal peptide 2+adjuvant (alum) or PBS+alum. Blood was collected for serologic analyses at 1, 2, 4, 6, and 8 weeks following each vaccination. Eight (8) weeks following the second vaccination, all animals were SIV infected and continuously exposed to Pneumocystis by co-housing. All animals were monitored monthly for Pneumocystis infection. FIG. 9B is a graph showing the mean plasma reciprocal endpoint titer (RET) of Pan-fungal peptide 2-specific immunoglobulin G (IgG) titer, as determined by enzyme-linked immunosorbent assay (ELISA). Timing of the first and second sets of immunizations are indicated by arrows. Pan-fungal peptide 2-immunized (vaccinated) macaques achieved a mean peak RET of 4×10⁵ two weeks after the second immunization was given (post vaccination 2 (2wpv2)) compared with sham-vaccinated animals (*p=0.019).

FIG. 10 is a graph showing chronic Pneumocystis colonization in Pan-fungal peptide 2-vaccinated and sham-vaccinated cohorts of Rhesus macaques. The graph illustrates the percentage of SIV-infected animals (Pan-fungal peptide 2-vaccinated (n=7) or sham-vaccinated (n=8)) that were chronically colonized with Pneumocystis (two or more consecutive months with positive nested PCR) over the course of the SIV infection (28 weeks post SIV inoculation) compared by Kaplan-Meier (*p=0.05). At 28 weeks post-infection, significantly fewer Pan-fungal peptide 2-immunized, SIV infected animals were chronically colonized with Pneumocystis compared with sham-immunized animals.

### DETAILED DESCRIPTION OF THE EMBODIMENTS

Featured herein are methods for treating or preventing infection by one or more fungal pathogens, or disease associated with infection by more than one fungal pathogen, in which the pathogens are of different etiologies. By way of specific example, disease-causing fungal pathogens include Pneumocystis, which colonizes lung tissue and causes severe pneumonia after infection; Aspergillus, a common mold, which causes aspergillosis, allergic reactions, lung infections and other health problems; Candida, which typically reside in the intestinal tract and mucous membranes and can cause thrush, infections and invasive candidiasis upon systemic infection, especially in those in poor health or with weak immune systems; and Cryptococcus, which can infect the lungs, where it can cause pneumonia-like illness, and the brain, where it can cause meningitis.

The described embodiments are based, at least in part, on the discovery that mammalian subjects, immunized with a non-naturally occurring peptide (referred to herein as a pan-fungal peptide) having sequence similarity to the Kexin (Kex or Kex1) protein generated antibodies that specifically bound Kex peptides of a number of distinct fungal pathogens, including Pneumocystis, Candida, Aspergillus, and Cryptococcus. Accordingly, pan-fungal peptides as described herein are useful for treating or preventing fungal infections by any one or more of Pneumocystis, Candida, Aspergillus and Cryptococcus.

Antibody-containing antiserum generated in response to administration of a non-naturally occurring pan-fungal Kex peptide, or a polynucleotide encoding the pan-fungal Kex peptide, can also serve as a treatment for infection and/or disease caused by more than one fungal pathogen and can provide immunity against multiple fungal pathogens in another or unrelated subject (i.e., a recipient subject) who receives the antiserum via a suitable mode and route of administration. It will be appreciated by the skilled practitioner that, as used herein, a subject from whom an antiserum is obtained or isolated is a “donor subject,” and a subject to whom the isolated antiserum is administered or provided is a “recipient subject.” In embodiments, a subject is a mammal, particularly a human being or a non-human primate. A recipient subject may be a patient or an individual in need of treatment for or protection from infection or disease caused by one or more of the Pneumocystis, Candida, Aspergillus and/or Cryptococcus fungal pathogens.

The production of such immunologically cross reactive antisera (and antibodies therein) produced in subjects (e.g., donor subjects) immunized with, or exposed to, a pan-fungal Kex peptide, or a polynucleotide encoding the pan-fungal Kex peptide, such as a non-naturally occurring consensus Kex1 peptide, that reacted with the Kex peptides of multiple fungal pathogens, such as Candida, Aspergillus and Cryptococcus fungal pathogens as described herein, was surprising and unexpected, particularly in view of the low amount of amino acid sequence identity (about 48%-70% variability) among the Kex peptides of the Pneumocystis, Candida, Aspergillus and Cryptococcus fungal pathogens, and in view of the overall amount of variability in the amino acid sequences (ranging from about 70% to 96% variability) between the Kex1 peptide of Pneumocystis and the Kex peptides of the Candida, Aspergillus and Cryptococcus fungal organisms.

Also embraced herein is an immunogenic composition comprising a pan-fungal peptide, or a polynucleotide encoding the pan-fungal peptide, that elicits a potent immune response in a subject following administration of the composition and the production of antiserum in the subject that contains one or more antibodies or antigen binding fragments thereof that reacts not only with the immunizing antigen, but also with a similar, but nonidentical, antigen found in other, distinct fungal pathogens.

One benefit of the described methods is the provision of treatment or prevention of infection by and disease associated with several different fungal pathogens using only one therapeutic agent, i.e., a composition comprising a non-naturally occurring pan-fungal Kex peptide, or a polynucleotide encoding the pan-fungal Kex peptide, or an antiserum (isolated antiserum) generated in response to administration of the non-naturally occurring pan-fungal Kex peptide or polynucleotide encoding the pan-fungal Kex peptide, that cross-protects against multiple fungal organisms and treats or prevents diseases and symptoms thereof, for example, pulmonary disease and poor pulmonary performance, associated with infection (and colonization) by the different fungal pathogens, e.g., at least two or more fungal pathogens. In an embodiment, the different fungal pathogens include Pneumocystis, Aspergillus, Candida, and Cryptococcus, particularly, at least two or more thereof. In one embodiment, a composition is provided that comprises a non-naturally occurring pan-fungal Kex peptide, or a polynucleotide encoding the pan-fungal Kex peptide, that elicits production of antibodies that specifically react with a Kex peptide derived from one or more of Pneumocystis (Pneumocystis jirovecii), Aspergillus (Aspergillus fumigatus), Candida (Candida albicans), or Cryptococcus (Cryptococcus neoformans). Another embodiment provides an antiserum produced in a subject immunized with a non-naturally occurring pan-fungal Kex peptide, or a polynucleotide encoding the pan-fungal Kex peptide, that contains antibodies that also specifically react with a Kex peptide derived from one or more of Pneumocystis (Pneumocystis jirovecii), Aspergillus (Aspergillus fumigatus), Candida (Candida albicans), or Cryptococcus (Cryptococcus neoformans). Accordingly, the antiserum is cross-protective (e.g., cross-reactive with Kex peptides in multiple fungal types) and affords treatment and/or protection against infection and/or disease associated with multiple fungal organisms when provided to another (e.g., unrelated) subject in need thereof. In an embodiment, the antiserum is an isolated antiserum. In an embodiment, the isolated antiserum is administered in a pharmaceutically acceptable composition.

The methods and compositions described herein offer economic, medical and practical benefits in the treatment and prevention of fungal infection and disease, such as pulmonary disease, or types of brain infections, associated with infection and colonization by different types of fungal pathogens

### Therapeutic Methods

The methods and compositions provided herein can be used to treat or prevent infection and/or associated disease caused by the fungal pathogens Pneumocystis, Aspergillus, Candida, and Cryptococcus, and, in particular, Pneumocystis hominis or jirovecii, Aspergillus fumigatus, Candida albicans, or Cryptococcus neoformans. The methods and compositions provided herein can provide immune protection in a subject against infection and disease caused by at least one, and particularly more than one, of these fungal organisms. The methods and compositions provided herein can immunize a recipient subject against infection by at least one and particularly by more than one of these fungal organisms. In general, an immunogenic composition comprising a non-naturally occurring pan-fungal Kex peptide, a polynucleotide encoding the pan-fungal Kex peptide, or an isolated antiserum containing one or more antibodies generated against the non-naturally occurring pan-fungal Kex peptide can be administered therapeutically and/or prophylactically to provide immunity against other pathogenic fungal organisms that express a Kex protein or peptide antigen. The methods include administering an immunologically effective amount of the immunogenic composition comprising a pan-fungal Kex peptide, a polynucleotide encoding the pan-fungal Kex peptide, an isolated antiserum, or immune serum or immune plasma, described herein to an individual, alone, or in a physiologically acceptable carrier, excipient, or diluent. In an embodiment, the immunogenic composition comprising a pan-fungal Kex peptide, a polynucleotide encoding the pan-fungal Kex peptide, or isolated antiserum is in a pharmaceutically acceptable composition.

Provided and described herein are methods of treating or preventing an infection by one or more fungal pathogens (e.g., one or more of Pneumocystis, Aspergillus, Candida, or Cryptococcus, more specifically, Pneumocystis hominis or jirovecii, Aspergillus fumigatus, Candida albicans, or Cryptococcus neoformans fungi), and/or diseases, disorders, or symptoms thereof, which comprise administering a therapeutically effective amount of an immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide as described herein to a recipient subject (e.g., a mammal such as a human patient) in need thereof. In an embodiment, the antiserum contains antibodies that specifically target the Kexin protein or a Kex peptide thereof to neutralize the activity of Kex proteinase. In an embodiment, the isolated antiserum allows the recipient subject to achieve and passively acquire protective immunity against multiple fungal pathogens.

In an embodiment, a method as described herein involves treating a subject suffering from or susceptible to an infection by Pneumocystis, Aspergillus, Candida, or Cryptococcus, or disease or symptom thereof caused by one or more of these fungal pathogens (e.g., pulmonary disease or COPD). The method includes the step of administering to the subject (e.g., a mammal or human patient) a therapeutic amount of an immunogenic composition comprising a pan-fungal Kex peptide, a polynucleotide encoding the pan-fungal Kex peptide, or an isolated antiserum generated against the non-naturally occurring pan-fungal Kex peptide that is sufficient to treat an infection, disease, disorder, or symptom thereof, caused by one or more different types of fungal organism under conditions such that the infection, disease, disorder, or symptom thereof, is treated. In an embodiment, the isolated antiserum is in a pharmaceutically acceptable composition.

Also provided and described are methods of treating or preventing infection by more than one type of fungal pathogen, and/or diseases or disorders or symptoms thereof, which comprise administering a therapeutically effective amount of an immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide as described herein (e.g., comprising one or more antibodies or an antigen binding fragment thereof), to a subject (e.g., a mammal such as a human). In various embodiments, the method prevents infection by more than one fungal pathogen selected from Pneumocystis, Aspergillus, Candida, or Cryptococcus in a subject susceptible to infection, disease or symptom thereof (e.g., COPD, lung/pulmonary disease, poor pulmonary function, or symptoms thereof). In an embodiment, the method includes the step of administering to a recipient mammal a therapeutic amount of an immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide sufficient to treat the infection, disease, disorder, or symptom thereof, under conditions such that the infection, disease disorder, or symptom thereof is treated. In an embodiment, the method includes the step of administering to a recipient mammal a prophylactic or preventive amount of an immunogenic composition comprising a non-naturally occurring pan-fungal Kex peptide, a polynucleotide encoding the pan-fungal Kex peptide, or an antiserum generated in response to the non-naturally occurring pan-fungal Kex peptide sufficient to prevent the infection, disease, disorder, or symptom thereof, under conditions such that the infection, disease disorder, or symptom thereof is prevented. In an embodiment, the isolated antiserum is in a pharmaceutically acceptable composition. In an embodiment, the recipient mammal is a human patient in need of treatment.

Treatment will be suitably administered to subjects, particularly humans, suffering from, having, susceptible to, or at risk for infection by, more than one fungal organism, in particular, more than one of Pneumocystis, Aspergillus, Candida, or Cryptococcus, and in particular, Pneumocystis hominis or jirovecii, Aspergillus fumigatus, Candida albicans, or Cryptococcus neoformans, or a disease, pathogenic condition, or symptom thereof. Determination of those subjects who are “at risk” can be made by any objective or subjective determination by a diagnostic test or opinion of a subject or health care provider (e.g., genetic test, enzyme test or assay, or protein marker (such as levels of anti-Kex antibodies, e.g., in serum), family history, and the like). The methods herein also include administering to the recipient subject (including a subject identified as in need of such treatment or as being at risk of infection) an effective amount of an anti-fungal pathogen immunogenic composition comprising a non-naturally occurring pan-fungal Kex peptide, a polynucleotide encoding the pan-fungal Kex peptide, or an antiserum generated in response to the non-naturally occurring pan-fungal Kex peptide and isolated from a donor subject as described herein. Identifying a subject in need of such treatment can involve the judgment of the recipient subject or a health care or medical professional and can be subjective (e.g., opinion) or objective (e.g., measurable by a test or diagnostic method). In an embodiment, the isolated antiserum is provided in a pharmaceutically acceptable composition.

In some aspects, methods of treating or preventing a fungal infection or fungal pathogen-associated disease or condition (e.g., pulmonary infection, pulmonary disease or disorder, pneumonia, COPD, and the like) in a subject are featured, in which the methods involve administering to a subject in need thereof an effective amount of an immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide and obtained from an individual who has produced an antibody immune response against the non-naturally occurring pan-fungal Kexin peptide as described herein, such that the subject is therapeutically and/or prophylactically treated against infection or disease associated with a different fungal pathogen.

Provided in another aspect are methods of treating or preventing fungal infection and/or disease in a patient who is receiving or who has received immune suppressive drugs or medication and who, as a result of drug-induced immune system suppression, is susceptible to or may become susceptible to (or at risk of) infection by a pathogenic fungus, such as one or more of the Pneumocystis, Aspergillus, Candida, or Cryptococcus fungal pathogens, either in or outside of a nosocomial environment. By way of example, such a patient may be preparing to undergo a transplant (a pre-transplant patient) or may have received a transplant (a post-transplant patient) and is administered one or more immunosuppressive drugs or medications (anti-rejection medications) and/or is otherwise treated with drugs to reduce the likelihood of rejection of the transplanted organ or tissue, thereby making the patient more vulnerable, susceptible to, or at risk of infection and/or disease caused by one or more of the Pneumocystis, Aspergillus, Candida, or Cryptococcus fungal pathogens. Patients having other types of diseases and conditions, such as rheumatoid arthritis or psoriasis, and the like, may also be administered medications having an immune suppressive effect to treat or manage their conditions and thus suffer from, or be at risk of, infection by one or more fungal pathogens. Non-limiting classes of immune suppressive drugs and medications include, for example, corticosteroids, such as prednisone (e.g., DELATSONE, ORASONE); budesonide (ENTOCORT EC), or prednisolone (MLLIPRED) calcineurin inhibitors, such as cyclosporine (NEORAL, SANDIMMUNE, SANGCYA); or tacrolimus (ASTAGRAF XL, ENVARSUS XR, PROGRAF); mTOR inhibitors, such as sirolimus (RAPAMUNE), everolimus (AFINITOR, ZORTRESS); Inosine Monophosphate Dehydrogenase (IMDH) inhibitors, such as azathioprine (AZASAN, IMURAN), leflunomide (ARAVA), mycophenolate (CELLCEPT, MYFORTIC); Biologics and monoclonal antibodies or monoclonal antibody-based antibodies or antigen binding fragments thereof, such as abatacept (ORENCIA); adalimumab (HUMIRA); anakinra (KINERET); certolizumab (CIMZIA); etanercept (ENBREL); golimumab (SIMPONI); infliximab (REMICADE); ixekizumab (TALTZ); natalizumab (TYSABRI); rituximab (RITIXAN); secukinumab (COSENTYX); tocilizumab (ACTEMRA); ustekinumab (STELARA); and vedolizumab (ENTYVIO). In an embodiment, the patient is to receive or has received a transplant of an organ selected from kidney, liver, heart, bone marrow, pancreas, lung, gall bladder, bladder, etc.

Immunogenic compositions comprising a non-naturally occurring pan-fungal Kex peptide, a polynucleotide encoding the pan-fungal Kex peptide, or antibodies directed against the non-naturally occurring pan-fungal Kex peptide (or antiserum containing such antibodies) can be administered to the patient who is receiving transplant rejection medication, or other immune suppressive medication, in an effective amount to heighten the immune response against infection by one or more, two or more, three or more, or all four of these fungal pathogens in the immune suppressed patient. In an embodiment, the patient receiving immune suppressing drugs can be evaluated and monitored during treatment with immune suppressive drugs for the presence of antibodies (and antibody titers) against one or more of the fungal pathogens by employing the methods and kits as described herein.

Optionally, an immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide as described herein may be administered in combination with one or more of any other treatment or therapy, e.g., anti-fungal therapies. For example, an immunogenic composition comprising a non-naturally occurring pan-fungal Kex peptide, a polynucleotide encoding the pan-fungal Kex peptide, or an isolated antiserum or immune plasma generated in response to the non-naturally occurring pan-fungal Kex peptide containing anti-Kex non-naturally occurring pan-fungal peptide antibody or an antigen binding fragment thereof as described herein may be administered in combination with other antibodies or antibody cocktails with anti-fungal activity (including, for example, immune plasma), or in combination with one or more drugs, for examples, one or more drugs having anti-fungal activity (e.g., trimethoprim-sulfamethoxazole, azithromycin-sulfamethoxazole, clarithromycin-sulfamethoxazole, atovaquone, sulfadoxine-pyrimethamine, erythromycin-sulfisoxazole, PS-15, and dapsone-trimethoprim, as well as intravenous pentamidine and clindamycin-primaquine), to provide protective immunity in the recipient against Pneumocystis, Aspergillus, Candida, or Cryptococcus fungal organisms. In an embodiment of any of the foregoing, the immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide is provided in a pharmaceutically acceptable composition.

In an embodiment of any of the foregoing aspects, the immunogenic composition comprising a non-naturally occurring pan-fungal Kex peptide, a polynucleotide encoding the pan-fungal Kex peptide, or the antiserum generated in response to the non-naturally occurring pan-fungal Kex peptide and isolated from a donor subject immunized, inoculated, or exposed to a Pneumocystis, Aspergillus, Candida, or Cryptococcus fungal pathogen, e.g., a Kex peptide expressed by such fungal pathogens) allows the recipient subject to acquire immune protection, including memory immune protection, against infection or disease caused by more than one of these fungal pathogens.

Methods for administering both single and combination therapies (e.g., concurrently or otherwise) are known to those skilled in the art and are described, for example, in Remington's Pharmaceutical Sciences, 12^{th} edition, Edited by E. W. Martin, Mack Publishing Co. In an embodiment, the antiserum provides a therapeutic, antibody-containing composition that treats infection or disease caused by one or more fungal pathogens as described herein. In another embodiment, the antiserum provides prophylactic, antibody-containing composition that prevents and protects against infection or disease caused by one or more fungal pathogens as described herein. In an embodiment, the isolated antiserum is in a pharmaceutically acceptable composition.

### Additional Methods

At present, there is a dearth of methods as well as reagents to determine if a patient who is asymptomatic for infection by one or more, two or more, three or more, or each of Pneumocystis, Aspergillus, Candida, or Cryptococcus fungal pathogens is susceptible, vulnerable, or at risk for infection by one or more of these pathogens. It is currently difficult to plate out these fungal organisms as they may be present in very low amounts, or they do not grow under the culture conditions available for assessing their presence in a subject. Consequently, it is difficult for a medical practitioner and the patient to know whether the patient is actually infected with one or more of Pneumocystis, Aspergillus, Candida, or Cryptococcus, or if they are likely to become infected, for example, after a medical procedure, surgery, or transplant.

It is also difficult to identify, qualify, or stratify patients who are or may be susceptible to infection by one or more fungal pathogen and to monitor patients for changes in susceptibility over time, e.g., during recovery from surgery or during immunosuppressive therapies, for example, following organ transplantation, or during other chemotherapy treatments, or for reduction or elimination of infection in a patient undergoing treatment for a fungal infection or associated disease over time. The methods described herein provide a viable solution for such medical needs. In addition, methods are provided that allow a patient to be treated with the appropriate or a more directed fungal therapy by stratifying patients based on whether they possess or do not possess antibodies specific for a particular fungal pathogen, thereby deterring infection, or based on whether they have or do not have anti-fungal Kex peptide antibodies that are specific for a given type of pathogenic fungus.

In an embodiment, a method is provided for detecting antibodies against a non-naturally occurring Kex protein or peptide in a sample obtained from a subject, in which the method comprises: (a) contacting a biological sample obtained from the subject with a non-naturally occurring Kex peptide and (b) detecting the binding of the non-naturally occurring Kex peptide to antibodies in the sample which specifically bind to the Kex peptide, said detection of binding being indicative of the presence of antibodies against the Kex peptide of the fungal organisms in the subject's sample. In an embodiment, the Kex peptide is attached to a solid support or substrate. In an embodiment, the binding is detected by performing an immunoassay, e.g., an enzyme linked immunosorbent assay.

In another embodiment, a method of monitoring or detecting antibodies to fungal organisms associated with infection in a subject who has undergone a transplant or who is to undergo a transplant procedure to determine, for example, whether the subject is protected or will be protected from infection by one or more fungal pathogens selected from Pneumocystis, Aspergillus, Candida, or Cryptococcus, in which the method comprises: (a) measuring at a first time point the level of antibodies that bind to a non-naturally occurring pan-fungal Kex protein or peptide in a sample obtained from the subject prior to undergoing transplant surgery; (b) measuring the levels of antibodies that bind to the non-naturally occurring pan-fungal Kex protein or peptide in a sample obtained from the subject at one or more time points after the subject has undergone transplant surgery; and (c) detecting that the subject sample contains a level of antibodies that specifically bind to the non-naturally occurring pan-fungal Kex peptide relative to a predetermined or threshold level or to a control level, wherein a high level of antibodies that bind to the non-naturally occurring pan-fungal Kex peptide in the subject's sample indicates that the subject has produced an immune response against the fungal organism. In an embodiment, antibodies detected in the subject's sample that bind to the non-naturally occurring pan-fungal Kex peptide may serve to protect the subject from infection by Pneumocystis, Aspergillus, Candida, or Cryptococcus, according to the present described methods and compositions.

Repeating the practice of the above-described method over time (at different time intervals or different time periods) allows monitoring of the subject's antibody levels against the non-naturally occurring pan-fungal Kex peptide (i.e., the levels of antibodies that bind the non-naturally occurring Kex peptide) and can inform the medical practitioner or clinician as to whether continued, new, or different treatment of the subject with an appropriate anti-fungal drug or therapy is needed or warranted, or whether no or less anti-fungal treatment is warranted, based on the measured titers of antibodies in the subject's sample.

Other embodiments also provide methods for detecting in a subject's biological sample, e.g., blood, serum, plasma, lymph, bronchoalveolar lavage fluid, the presence of antibodies that bind a non-naturally occurring pan-fungal Kex peptide, wherein the levels of antibodies against the non-naturally occurring pan-fungal Kex peptide in the biological sample are determined simultaneously. For example, in one embodiment, the method comprises: (a) contacting a biological sample obtained from the subject with a non-naturally occurring pan-fungal Kex peptide that selectively binds to a plurality of antibodies in the subject's sample for a period of time sufficient to form bound Kex peptide-antibody complexes; (b) detecting binding of the Kex peptides to the plurality of antibodies in the subject's sample, thereby determining the levels of antibodies to the Kex peptides in the sample; and (c) comparing the levels of the plurality of antibodies in the sample with predetermined threshold values, wherein levels of antibodies that bind to at least one of the plurality of Kex peptides above or below the predetermined threshold values indicates, for example, that the subject has an antibody titer and has generated an immune response against the Kex peptide derived from one or more of the fungal organisms. Accordingly, the subject having a measured antibody response to the Kex peptide is protected from infection and disease associated with one or more of the Pneumocystis, Aspergillus, Candida, or Cryptococcus fungal organisms.

In another embodiment, methods for assessing antibodies that bind to the Kex peptide of one or more of the fungal organisms Pneumocystis, Aspergillus, Candida, or Cryptococcus in a subject are provided, in which the methods comprise: (a) contacting a biological sample obtained from the subject with a composition comprising one or more (a plurality of) Kex peptides derived from Pneumocystis, Aspergillus, Candida, or Cryptococcus for a period of time sufficient to form antibody-Kex peptide complexes; (b) detecting binding of the plurality of the Kex peptides to antibodies in the sample, thereby detecting the level or titer of anti-Kex peptide antibodies in the sample; and (c) comparing the level or titer of the anti-Kex peptide antibodies in the biological sample with predetermined threshold values or control values, wherein levels of at least one of the anti-Kex peptide antibodies above or below the predetermined threshold values indicates that the subject has or does not have, respectively, an adequate immune response (antibody response) to prevent infection by the one or more fungal organisms.

In another embodiment, a composition is provided, which comprises a solid substrate and a plurality of Kex peptides derived from one or more of Pneumocystis, Aspergillus, Candida, or Cryptococcus immobilized on the substrate. In an embodiment, a Kex peptide from each of Pneumocystis, Aspergillus, Candida, or Cryptococcus is immobilized at a different, indexable, location on the substrate. In other embodiments, a mixture of Kex peptides from two or more, three or more, or all four of Pneumocystis, Aspergillus, Candida, or Cryptococcus is immobilized at a different, indexible, location on the substrate. The binding of anti-Kex peptide antibodies from a sample obtained from a subject previously vaccinated with a non-naturally occurring pan-fungal Kex peptide can be measured or detected by measuring or detecting complexes of the anti-Kex peptide antibodies bound to the Kex peptides localized on the substrate. In an embodiment, the composition is contained in a kit for performing an immunoassay to detect and/or measure the antibody-peptide complexes, as well as determine or measure the level or amount of antibody present in the subject's sample.

### Antibodies

As described herein, antisera comprising antibodies that specifically bind a non-naturally occurring Kexin peptide and that cross-react with a Kexin peptide of one or more different fungal organisms, such as one or more of Pneumocystis, Aspergillus, Candida, or Cryptococcus, to provide immune protection against infection and disease caused by two or more of these fungal pathogens are useful in therapeutic methods. For example, isolated antiserum containing antibodies that target and/or inhibit or neutralize the activity of the non-naturally occurring pan-fungal Kexin peptide and that cross-reacts with two or more of these fungal pathogens is particularly useful in the methods as described herein. In particular embodiments, methods of using isolated antiserum (or immune plasma) comprising antibodies, or antigen binding fragments thereof, that specifically bind the non-naturally occurring pan-fungal Kex peptide and that cross-react with Pneumocystis, Aspergillus, Candida, or Cryptococcus Kexin polypeptides are provided for the treatment or prevention of infection by these fungal pathogens and/or their associated diseases and conditions, such as pulmonary diseases and disorders of various types, pneumonia, COPD, invasive pulmonary aspergillosis (IPA), etc.

Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of an intact antibody, clear more rapidly from the circulation, and may have less nonspecific tissue binding than an intact antibody (Wahl et al., J. Nucl. Med. 24:316-325 (1983)). The antibodies may comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, (1995)), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody, because small molecules of ~60-100 kDa in size provide faster blood clearance and rapid tissue uptake. See, e.g., Power et al., (Generation of recombinant multimeric antibody fragments for tumor diagnosis and therapy, Methods Mol Biol, 207, 335-50, (2003); and Wu et al., Anti-carcinoembryonic antigen (CEA) diabody for rapid tumor targeting and imaging, Tumor Targeting, 4, 47-58, (1999)).

Various techniques for making and using unconventional antibodies have been described. Bispecific antibodies produced using leucine zippers are described by Kostelny et al. (J. Immunol. 148(5):1547-1553, (1992)). Diabody technology is described by Hollinger et al. (Proc. Natl. Acad. Sci. USA 90:6444-6448, (1993)). Another strategy for making bispecific antibody fragments using single-chain Fv (sFv) diners is described by Gruber et al. (J. Immunol. 152:5368, (1994)). Trispecific antibodies are described by Tutt et al. (J. Immunol. 147:60, (1991)). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, (1988)). See, also, U.S. Pat. Nos. 5,091,513, 5,132,405 and 4,956,778; and U.S. Patent Publication Nos. 20050196754 and 20050196754.

In various embodiments, an antiserum (isolated antiserum) contains antibodies, or antigen binding fragments thereof, that specifically bind the non-naturally occurring Kex peptide that are monoclonal or polyclonal. Also encompassed are methods of obtaining or isolating the antibodies from immune serum (antiserum) or immune plasma and producing hybrid or chimeric antibodies therefrom. In such hybrid or chimeric antibodies one pair of heavy and light chains is obtained from a first antibody, while the other pair of heavy and light chains is obtained from a different second antibody. Such hybrids or chimeric antibodies may also be formed using humanized heavy and light chains. Methods for isolating antibodies and producing hybrid or chimeric antibodies are known and practiced by those having skill in the art.

In general, intact antibodies are said to contain “Fc” and “Fab” regions. The Fc regions are involved in complement activation and are not involved in antigen binding. An antibody from which the Fc region has been enzymatically cleaved, or which has been produced without the Fc region, designated an “F(ab′)₂” fragment, retains both antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies (and immune serum or plasma containing antibodies) can be produced or generated by any of the methods known in the art utilizing soluble polypeptides, or immunogenic fragments thereof, (e.g., a Kex pan-fungal peptide) as an immunogen. One method of obtaining antibodies is to immunize suitable host animals or subjects with an immunogen, a polynucleotide encoding the pan-fungal Kex peptide, and to follow standard procedures for polyclonal or monoclonal antibody production. In brief, the immunogen will facilitate presentation of the immunogen (or immunogenic fragments of the immunogen) on the cell surface. Immunization of a suitable host can be carried out in a number of ways. By way of example, nucleic acid sequences encoding immunogenic pan-fungal Kexin peptides can be provided to the host in a delivery vehicle (or a molecular expression construct) that is taken up by immune cells of the host. The cells will, in turn, process and appropriately express the pan-fungal Kex peptide in a manner that generates an immunogenic response in the host. In embodiments, non-naturally occurring pan-fungal Kex peptides may be expressed by the delivery vehicle or expression construct. In other exemplary embodiments, nucleic acid sequences encoding non-naturally occurring pan-fungal Kex peptides may be expressed in cells in vitro, and the expressed, recombinant pan-fungal Kex peptide products may be isolated and used as immunogens to raise anti-Kex peptide antibodies and to generate an anti-Kex antiserum in a suitable immunized host.

Alternatively, antibodies against non-naturally occurring pan-fungal Kex peptides may, if desired, be derived from an antibody phage display library. A bacteriophage is capable of infecting and reproducing within bacteria, which can be engineered, when combined with human immunoglobulin (antibody) genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from an immunized host. Antibody purification methods include, without limitation, salt precipitation (for example, with ammonium sulfate), ion exchange chromatography (for example, on a cationic or anionic exchange column, preferably run at neutral pH and eluted with step gradients of increasing ionic strength), gel filtration chromatography (including gel filtration HPLC) and chromatography on affinity resins such as protein A, protein G, hydroxyapatite, or anti-immunoglobulin.

In certain aspects, antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can, in turn, be obtained from the hybridoma that produces the antibody, and then the antibody may be produced synthetically or recombinantly from these nucleic acid sequences. To produce large amounts of antibody, it is generally more convenient to obtain an ascites fluid. The method of raising ascites fluid generally comprises injecting hybridoma cells into an immunologically naive histocompatible or immunotolerant mammal, especially a mouse. The mammal may be primed for ascites production by prior administration of a suitable composition (e.g., Pristane). Ascites fluid containing antibodies, typically in high concentration, can be obtained from the peritoneal fluid of the animal that harbors the injected hybridoma cells.

Monoclonal antibodies (Mabs can also be “humanized” by methods known in the art. “Humanized” antibodies are antibodies in which at least part of the sequence has been altered from its initial form to render it more like immunoglobulins derived from a human source. Techniques to humanize antibodies are particularly useful when antibodies are generated in a non-human animal (e.g., mice, rats). Nonlimiting examples of methods for humanizing a murine antibody are provided in U.S. Pat. Nos. 4,816,567, 5,530,101, 5,225,539, 5,585,089, 5,693,762 and 5,859,205.

In an embodiment of the foregoing, one or more antibodies or antigen binding fragments thereof generated against non-naturally occurring Kex peptides can be used in a pharmaceutical composition alone or in combination to provide immune protection against disease or infection caused by one or more of these fungal pathogens in a subject in need thereof. Such antibodies may be isolated or purified from an antiserum as described herein, or they may be generated, e.g., by recombinant molecular biology techniques, purified and formulated for pharmaceutical use in a subject in need. Such a formulation of antibodies may have immune protective properties similar to those afforded by an isolated antiserum comprising anti-fungal Kex peptide antibodies as described herein.

### Vaccines

A vaccine is a biological preparation that provides active, acquired immunity (e.g., protective immunity) in a subject to a particular disease. A vaccine typically contains an agent that resembles a disease-causing pathogenic agent, e.g., a microorganism, a fungus, etc., and is often made from a weakened or killed form of the agent, or a toxin or surface protein or peptide of the agent. After administration of the vaccine to a subject, the agent is expressed and recognized as foreign (or “non-self”) to the subject and stimulates the subject's immune system to mount an immune response (a B cell (antibody) and/or a T cell (cellular) immune response) and to destroy the agent. In addition, cells (e.g., B cells) of the immune system that are exposed to the vaccinating agent retain a memory of the agent, such that the agent is recognized and destroyed by the memory cells upon a later or subsequent encounter. Vaccines can be prophylactic (e.g., to prevent or ameliorate the effects of a future infection by a pathogen), or therapeutic (e.g., to treat disease or infections caused by or associated with pathogens or disease-causing agents upon or after a subject has been infected with or encountered a pathogen).

While many vaccines are prepared from an attenuated version of a pathogen or from inactivated disease-causing organisms, or a suitable part of such pathogens or organisms, such as a toxin, protein/peptide, or deleterious enzyme, the antigen to which the immune system responds frequently constitutes a relatively small number of amino acids, such as a peptide (e.g., a non-naturally occurring pan-fungal Kex peptide). A protein or peptide part of a pathogen may constitute a vaccine. A peptide vaccine is any peptide which serves to immunize an organism (elicit an immune response or a protective immune response, such as an antibody (B cell) response and/or an immune cell (T cell) response in the immunized organism) against a pathogen. In embodiments, the peptide antigen may be a non-naturally occurring pan-fungal Kex peptide. In an embodiment, a vaccine comprising a non-naturally occurring Kex peptide antigen may be used to provide immune protection against each of the other fungal organisms following administration to a recipient subject in need.

For non-attenuated vaccines, the peptide sequences that trigger a protective immune response are identified, and synthetic (or recombinantly-produced) versions of the peptides are employed as the vaccine substance. Because they are non-naturally occurring and synthetic, peptide vaccines pose little to no risk of mutation or reversion, and little or no risk of contamination by pathogenic or toxic substances. Moreover, chemical manipulation or modification of the peptide structure may result in increased stability and decreased unwanted side effects or adverse effects that may be associated with a native protein or peptide sequence.

Synthetically or recombinantly produced peptide antigens can be readily prepared in large amounts as components of vaccines. Such substances may also expose parts of a protein antigen that are not recognized by the immune system during a natural infection, possibly as a result of masking or post-translational modifications of proteins. Sequencing new strains and serotypes of microorganisms, fungal pathogens and other pathogenic organisms allows for rapid modification of peptide antigens to generate strain-specific immune responses, particularly against an antigenic epitope that is recognized and targeted by antibodies and cells of the host's or recipient's immune system. In some cases, modelling of three-dimensional epitopic or antigenic sites of a pathogen may be employed to generate synthetically the correct epitopic or antigenic site(s) on peptide antigens.

In an aspect, a vaccine (or an immunogenic composition) is provided, which comprises a synthetically (recombinantly) produced peptide, i.e., a pan-fungal Kexin peptide, that is nonidentical, but immunologically targetable, among several different types of fungal pathogens (e.g., the Pneumocystis, Aspergillus, Candida and Cryptococcus fungal pathogens) and is useful for treating or preventing infection or disease caused by or associated with the multiple fungal pathogens after administration (immunization) to a subject. In an embodiment, a peptide vaccine or immunogenic composition from a non-naturally occurring pan-fungal Kex peptide, when used to immunize an individual, elicits an immune response in the form of the production of antiserum (or immune plasma) containing cross-reactive antibodies which protect against all of the etiologically distinct fungal pathogens Pneumocystis, Aspergillus, Candida and Cryptococcus, and in particular, Pneumocystis hominis, Aspergillus fumigatus, Candida albicans and Cryptococcus neoformans. Accordingly, an antiserum or immune plasma generated by a vaccine or immunogenic composition comprising a non-naturally occurring pan-fungal Kex peptide may be used as a sole therapeutic or protective agent needed to treat or prevent infection or disease caused by or associated with more than one different fungal pathogen, namely, the Pneumocystis, Aspergillus, Candida and/or Cryptococcus fungal pathogens, and in particular, infection or disease caused by or associated with Pneumocystis hominis, Aspergillus fumigatus, Candida albicans and/or Cryptococcus neoformans. In an embodiment, the antiserum generated by such as peptide vaccine is isolated. In an embodiment, the isolated antiserum is used in a pharmaceutical composition.

In some aspects, a genetic vaccine is provided. A genetic vaccine is any vaccine that comprises a polynucleotide sequence encoding an immunogen, wherein the immunogen, once expressed, serves to immunize an organism. Administration of a genetic vaccine elicits an immune response or a protective immune response, such as an antibody (B cell) response and/or an immune cell (T cell) response in the immunized organism against a pathogen after the immunogen is expressed in a cell. In some embodiments, the genetic vaccine provides a polynucleotide sequence encoding a Kex peptide, such as a pan-fungal or wildtype peptide. In some embodiments, the peptide is a non-naturally occurring peptide. In some embodiments, the genetic vaccine provides a polynucleotide sequence encoding more than one Kex peptide. In some embodiments, the polynucleotide encoding the Kex peptide resides in a vector having elements, such as promoters and enhancers, to facilitate expression of the encoded Kex peptide. The nucleic acid in a genetic vaccine may be integrated into the subject's genome, wherein expression of the immunogen may be driven by an endogenous promoter or a promoter encoded by the inserted nucleic acid. The polynucleotide encoding the immunogen in a genetic vaccine may be a DNA polynucleotide or a RNA polynucleotide. The polynucleotide may include a nucleotide analog, which can inhibit degradation of the polynucleotide.

### Pharmaceutical Compositions

Also featured herein are methods for treating or preventing infection and disease associated with infection of a subject by one or more fungal pathogens. The methods include administering to a subject in need thereof an immunologically effective amount of an immunogenic composition comprising a non-naturally occurring pan-fungal Kex peptide, a polynucleotide encoding the pan-fungal Kex peptide, or an isolated antiserum generated in response to the non-naturally occurring pan-fungal Kex peptide which cross-treats and/or protects the subject from infection by and/or disease associated with infection by and/or disease associated with one or more different fungal pathogens selected from Pneumocystis, Aspergillus, Candida, or Cryptococcus. In an embodiment, the isolated antiserum is used in a pharmaceutical composition.

Typically, the carrier or excipient for an immunogenic composition or vaccine as described herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intranasal, and the like. Such methods also include administering an adjuvant, such as an oil-in-water emulsion, a saponin, a cholesterol, a phospholipid, a CpG, a polysaccharide, variants thereof, and a combination thereof, with a composition as described herein. Optionally, a formulation for prophylactic administration also contains one or more adjuvants for enhancing the immune response to an antigen or immunogen, such as a pan-fungal Kex peptide antigen or immunogen. Suitable adjuvants include, without limitation, complete Freund's adjuvant, incomplete Freund's adjuvant, saponin, alpha-galactosylceramide (α-GC), mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59. In an embodiment, the isolated antiserum is used in a pharmaceutical composition.

The administration of an immunogenic composition comprising a non-naturally occurring pan-fungal Kex peptide, a polynucleotide encoding the pan-fungal Kex peptide, or an antiserum, such as an isolated antiserum, monoclonal or polyclonal antibodies against the non-naturally occurring pan-fungal Kexin protein or peptide as a therapeutic for the treatment or prevention of an infection by a fungal pathogen as described herein or a fungal pathogen-associated disease or condition (e.g., pulmonary infection or disease, poor pulmonary function, COPD, pneumonia, etc.) may be by any suitable means that results in a concentration of the therapeutic that, combined with other components, if desired, is effective in ameliorating, reducing, eliminating, abating, or stabilizing disease or disease symptoms in a subject. The therapeutic may be administered systemically, for example, formulated in a pharmaceutically-acceptable composition or buffer such as physiological saline. Preferable routes of administration include, for example, subcutaneous, intravenous, intraperitoneally, intramuscular, intrathecal, or intradermal injections that provide continuous, sustained levels of the therapeutic in the subject. The amount of the therapeutic to be administered varies depending upon the manner of administration, the age and body weight of the subject, and with the clinical symptoms of the fungal infection or associated disease. Generally, amounts will be in the range of those used for other agents used in the treatment of pulmonary disease or dysfunction, although in certain instances lower amounts may be suitable because of the increased range of protection and treatment afforded by the therapeutic. A composition is administered at a dosage that ameliorates, decreases, diminishes, abates, alleviates, or eliminations the effects of the fungal pathogen infection or disease (e.g., pulmonary infection and disease and the symptoms thereof) as determined by a method known to one skilled in the art. In an embodiment, the isolated antiserum is administered or provided to a recipient subject at or near a site of the infection or colonization by the pathogenic fungal organism or organisms.

In embodiments, a therapeutic or prophylactic treatment agent may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart; (v) formulations that allow for convenient dosing, such that doses are administered, for example, once every one or two weeks; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, an isolated antiserum may be formulated with one or more additional components for administration to a subject.

Any of a number of strategies can be pursued to obtain controlled release in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

A pharmaceutical composition may be administered parenterally by injection, infusion or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, noted supra.

Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease or dysfunction, such as pulmonary disease or dysfunction, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, a pharmaceutical composition comprising an active therapeutic (e.g., an immunogenic composition comprising a non-naturally occurring pan-fungal Kex peptide, a polynucleotide encoding the pan-fungal Kex peptide, or an isolated anti-fungal antiserum generated in response to the non-naturally occurring pan-fungal Kex peptide as described herein) is formulated for intravenous delivery, e.g., intravenous, injection, or intrathecal delivery. In an embodiment, the antiserum is an isolated antiserum. To prepare such a composition, the suitable therapeutic(s) are dissolved or suspended in a parenterally acceptable liquid vehicle, excipient, or solvent. Among acceptable vehicles and solvents that may be employed are, for example, water; water adjusted to a suitable pH by the addition of an appropriate amount of hydrochloric acid, sodium hydroxide or a suitable buffer; 1,3-butanediol; Ringer's solution; and isotonic sodium chloride solution and dextrose solution. The aqueous formulation may also contain one or more preservatives (e.g., methyl, ethyl or n-propyl p-hydroxybenzoate). In cases in which one of the agents is only sparingly or slightly soluble in water, a dissolution enhancing or solubilizing agent can be added, or the solvent may include 10-60% w/w of propylene glycol or the like.

Kits and Compositions for Detecting and/or Quantifying Antibodies that React with Fungal-Derived Kex Peptides

In another embodiment, kits and compositions are provided that advantageously allow for the detection and/or quantification of the presence of antibodies directed against the Kex protein or peptide of one or more of Pneumocystis, Aspergillus, Candida, or Cryptococcus fungal pathogens, or the levels of such one or more antibodies that may be present, in a subject's sample (e.g., blood or serum). In an embodiment, the subject is a human patient. In an embodiment, the patient has undergone a transplant, e.g., an organ or tissue transplant, or is to undergo a transplant, and thus may be at higher risk for infection by one or more fungal pathogens. In an embodiment, the transplant patient, or the patient to undergo a transplant, is immunosuppressed and/or is otherwise treated with drugs to reduce the likelihood of rejection of the transplanted organ or tissue, thereby making the patient more vulnerable or susceptible to infection and/or disease caused by one or more of the Pneumocystis, Aspergillus, Candida, or Cryptococcus fungal pathogens. In an embodiment, the patient has received, or is to receive, a transplant of an organ selected from kidney, liver, heart, bone marrow, pancreas, lung, etc.

Such kits as described herein fulfill a long-felt need in the art for detecting or qualifying whether any patient, but particularly a transplant patient, has adequate levels (titer) of anti-fungal pathogen antibodies to ensure that the patient does not become infected with one or more fungal pathogens as described herein, for example, during a hospital stay, or during or following a medical procedure or treatment (e.g., surgery or transplant), performed either on in-patient or an out-patient basis. At present, because of a lack of appropriate reagents and assays, it is difficult to assess whether a patient who is to undergo a medical procedure or surgery, in particular, an immunosuppressed patient who is to undergo a transplant procedure, or a patient who is to initiate other immunosuppressive therapies, will contract a fungal infection, e.g., infection and/or disease caused by one or more of the Pneumocystis, Aspergillus, Candida, or Cryptococcus fungal pathogens following or during immunosuppressive therapies and treatments. The use of a kit with which a patient's sample can be tested to determine if the patient has an antibody titer against one or more of these fungal pathogens (e.g., a high or a low antibody titer against one or more of the fungal pathogens) would greatly enhance the success of the patient's post-surgical or post-transplant recovery and directed treatment. For example, if, following testing of a patient's sample (e.g., a blood or serum sample from a transplant patient) using a kit as described herein, the patient is determined to have a low, negligible, or no antibody (antiserum) titer against one or more of the fungal pathogens, in particular, against the Kex peptide of one or more of the fungal pathogens, it could be surmised that the patient would not be naturally protected against a possible or real infection by one or more of the Pneumocystis, Aspergillus, Candida, or Cryptococcus.

A kit as described herein would allow the tester and the patient to determine and know if the patient's sample (serum sample) contains antibodies against one or more, two or more, three or more, or four of Kex protein/peptide of the Pneumocystis, Aspergillus, Candida, or Cryptococcus organisms. Should the results obtained from the use of the kit indicate that the patient has no specific anti-fungal Kex peptide antibodies, or a low titer of such antibodies (e.g., no specific anti-fungal Kex peptide antibodies in serum), directed to a specific anti-fungal Kex peptide, the patient would be identified as potentially vulnerable or susceptible to infection to (a) particular fungal pathogen(s) and could then be administered the appropriate anti-fungal treatment for the specific fungal pathogen(s) against which the patient has no, or negligible, specific antibodies, or a reduced antibody titer. In an embodiment, the patient is administered a prophylactic anti-fungal treatment or therapy. In an embodiment, the treatment comprises administering to the patient an appropriate drug or medication that is best designed to treat infection or disease associated with infection by a specific fungal pathogen or by two or more fungal pathogens, namely, Pneumocystis, Aspergillus, Candida, or Cryptococcus. In an embodiment, the treatment comprises administering to the patient a composition as described herein comprising a non-naturally occurring pan-fungal Kex peptide or a polynucleotide encoding the pan-fungal Kex peptide to generate a cross-reactive (cross-protective) antibody immune response in the patient, thereby reducing or eliminating one or more of the Pneumocystis, Aspergillus, Candida, or Cryptococcus organisms associated with the infection. Antibodies produced against a non-naturally occurring pan-fungal Kex peptide of one of the fungal organisms can recognize a Kex peptide of the other fungal organisms as described herein, thereby conferring protection (cross-protection) against more than one of the fungal organisms in the patient.

In an embodiment, a kit is provided for detecting, or qualifying the levels of, antibodies directed against the non-naturally occurring pan-fungal Kex protein or peptide and that cross-react with one, two, three, or four of Pneumocystis, Aspergillus, Candida, or Cryptococcus organisms in a patient sample, in which the kit comprises a substrate having attached thereto a non-naturally occurring pan-fungal Kex peptide derived from one or more, two or more, three or more, or each of Pneumocystis, Aspergillus, Candida, or Cryptococcus fungal organisms for measuring the level of antibodies in the sample which bind or immunologically react with the peptide, onto which a biological sample obtained from a patient is applied; and a labeled detection molecule for detecting and measuring the level of antibodies that bind to the Kex peptides on the substrate. In an embodiment, detecting anti-fungal Kex peptide antibodies in the sample or the measuring the level of such antibodies present in the patient's sample is compared to a positive and/or a negative control. In an embodiment, detecting anti-fungal Kex peptide antibodies in the sample or the measuring the level of such antibodies present in the patient's sample is compared to a cutoff value. In an embodiment, the substrate has attached thereto a non-naturally occurring pan-fungal Kex peptide and a Kex peptide derived from each of Pneumocystis, Aspergillus, Candida, and Cryptococcus. In an embodiment, the substrate has attached thereto a non-naturally occurring pan-fungal Kex peptide and Kex peptides derived from one of Pneumocystis, Aspergillus, Candida, and Cryptococcus. In an embodiment, the substrate has attached thereto non-naturally occurring pan-fungal Kex peptide and Kex peptides derived from Pneumocystis and Aspergillus. In an embodiment, the substrate has attached thereto anon-naturally occurring pan-fungal Kex peptide and Kex peptides derived from Pneumocystis and Candida. In an embodiment, the substrate has attached thereto a non-naturally occurring pan-fungal Kex peptide and Kex peptides derived from Pneumocystis and Cryptococcus. In an embodiment, the substrate has attached thereto a non-naturally occurring pan-fungal Kex peptide and Kex peptides derived from Candida and Aspergillus. In an embodiment, the substrate has attached thereto a non-naturally occurring pan-fungal Kex peptide and Kex peptides derived from Cryptococcus and Aspergillus. In an embodiment, the substrate has attached thereto a non-naturally occurring pan-fungal Kex peptide and Kex peptides derived from Candida and Cryptococcus. In an embodiment, the substrate has attached thereto a non-naturally occurring pan-fungal Kex peptide and Kex peptides derived from Pneumocystis, Aspergillus and Candida. In an embodiment, the substrate has attached thereto a non-naturally occurring pan-fungal Kex peptide and Kex peptides derived from Pneumocystis, Aspergillus and Candida. In an embodiment, the substrate has attached thereto a non-naturally occurring pan-fungal Kex peptide and Kex peptides derived from Pneumocystis, Aspergillus and Cryptococcus. In an embodiment, the substrate has attached thereto a non-naturally occurring pan-fungal Kex peptide and Kex peptides derived from Pneumocystis, Cryptococcus and Candida. In an embodiment, the substrate has attached thereto a non-naturally occurring pan-fungal Kex peptide and Kex peptides derived fromAspergillus, Cryptococcus and Candida. In an embodiment, the pan-fungal Kex peptides are recombinantly produced. In an embodiment, the detection of antibodies in the sample that bind to the fungal-derived Kex peptide is performed using an immunoassay, such as an ELISA. In an embodiment, the ELISA detects a complex between a Kex peptide bound to an anti-fungal Kex peptide antibody present in the sample. In an embodiment, the detection of antibodies in the sample that bind to the fungal-derived Kex peptide is performed using an immunosorbent assay, by immunoprecipitation, by immunoblotting, or a combination thereof.

Also provided are kits comprising reagents that allow for assessing, measuring, evaluating or detecting antibodies directed against a non-naturally occurring Kex peptide. Such antibodies may be contained in a biological sample obtained from a subject undergoing testing, assessment, or evaluation using the kit. In particular, the biological sample may be a blood, serum, plasma, urine, cerebrospinal fluid, sputum, bronchiolar lavage, tears, saliva, or semen sample, or tissue or cell sample obtained from a subject. In particular, the reagents of the kit comprise non-naturally occurring Kex peptides.

In a specific embodiment, the kit is provided as an enzyme linked immunosorbent assay (ELISA) kit comprising a non-naturally occurring pan-fungal Kex peptide. In other embodiments, the provided kit allows for the detection of cross-reactive antibodies, wherein the antibodies are produced by immunization with a non-naturally occurring pan-fungal Kex peptide. In such embodiments, the kit is provided as an ELISA kit comprising the Kex peptides of Pneumocystis, Aspergillus, Candida, or Cryptococcus attached to a solid support or substrate. The peptides attached to the substrate thus perform as “capture” reagents that bind to antibodies present in the sample obtained from a subject undergoing testing. By way of example, the ELISA kit may comprise a solid support, such as a chip, microtiter plate comprising many wells (e.g., a 96-well plate), bead, or resin having the peptide capture reagents attached thereon. In one embodiment, the kit comprises a Kex peptide derived from each of Pneumocystis, Aspergillus, Candida, or Cryptococcus as described herein attached independently to discrete areas or components of solid substrates or supports, for example, the Kex peptides of each fungal organism are attached to separate and discrete wells of a microtiter plate or are independently attached to beads to produce populations of beads having the Kex peptides from each of Pneumocystis, Aspergillus, Candida, or Cryptococcus attached. In another embodiment, the kit comprises a combination or mixture of the Kex peptides derived from Pneumocystis, Aspergillus, Candida, or Cryptococcus attached to an area or component of the solid substrate or support, for example, the Kex peptides of all of Pneumocystis, Aspergillus, Candida, or Cryptococcus are attached to a single well of a microtiter plate or to a single bead. In a further embodiment, the kit comprises a combination of one, two or more, three or more, or four of the Kex peptides derived from Pneumocystis, Aspergillus, Candida, or Cryptococcus attached to a given area of a solid substrate or support, such as a single well of a microtiter plate.

In embodiments, in the ELISA platform, a well of a microtiter plate may have attached thereto a non-naturally occurring pan-fungal Kex peptide, an Aspergillus Kex peptide, a Candida Kex peptide, or a Cryptococcus Kex peptide. In the ELISA platform, a well of a microtiter plate may have attached thereto a Pneumocystis Kex peptide and an Aspergillus Kex peptide. In the ELISA platform, a well of a microtiter plate may have attached thereto a Pneumocystis Kex peptide and a Candida Kex peptide. In the ELISA platform, a well of a microtiter plate may have attached thereto a Pneumocystis Kex peptide and a Cryptococcus Kex peptide. In the ELISA platform, a well of a microtiter plate may have attached thereto an Aspergillus Kex peptide and a Candida Kex peptide. In the ELISA platform, a well of a microtiter plate may have attached thereto an Aspergillus Kex peptide and a Cryptococcus Kex peptide. In the ELISA platform, a well of a microtiter plate may have attached thereto a Candida Kex peptide and a Cryptococcus Kex peptide. In the ELISA platform, an individual well of a microtiter plate may have attached thereto a Pneumocystis Kex peptide, an Aspergillus Kex peptide and a Candida Kex peptide. In the ELISA platform, an individual well of a microtiter plate may have attached thereto aPneumocystis Kex peptide, an Aspergillus Kex peptide and a Cryptococcus Kex peptide. In the ELISA platform, an individual well of a microtiter plate may have attached thereto a Pneumocystis Kex peptide, a Candida Kex peptide and a Cryptococcus Kex peptide. In the ELISA platform, an individual well of a microtiter plate may have attached thereto an Aspergillus Kex peptide, a Candida Kex peptide and a Crytococcus Kex peptide.

The kit may further comprise a means for detecting the peptides or any antibodies bound thereto, e.g., detectable antibodies, a secondary antibody-signal complex, such as horseradish peroxidase (HRP)-conjugated goat anti-rabbit IgG antibody or tetramethyl benzidine (TMB) as a substrate for HRP.

In another embodiment, the kit may be provided as an immunochromatography strip comprising a membrane on which the one, two, three, or four Kex peptides are immobilized, either at discrete loci on the membrane or in combination at one locus of the membrane, and a means for detecting the binding of antibody in a test sample, e.g., detectably labeled peptides, or gold particle bound secondary antibodies, in which the membrane may be a nitrocellulose-based (NC) membrane, a PVDF membrane, or other suitable type of membrane used in the art. The kit may comprise a plastic plate or substrate onto which a sample is applied and immobilized detection agents, such as detectably labeled Kex peptides, e.g., gold particle-bound peptides temporally spaced and immobilized on the substrate, e.g., a glass fiber filter or a nitrocellulose membrane, or a labeled detection agent that can detect a complex of antibody bound to Kex peptide in one or more bands on the substrate. In such a platform, a continuous capillary flow of sample, e.g., blood or serum, is maintained over the detection reagents immobilized on the substrate such that sample antibody bound to labeled Kex peptide or sample antibody complexed to Kex peptide reagent may be detected. In general, ELISA assays and immunosorbent assays, including ELISA membrane-based immunosorbent assays, as well as variations of these assays, are known and practiced by those having skill in the art.

Solid or solid phase substrates, or carriers, that can be effectively used in such assays are well known to those of skill in the art and include, for example, 96-well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in U.S. Patent Application Publication No. US 2010/0093557 A1. Methods for the automation of immunoassays are well known in the art and include, for example, those described in U.S. Pat. Nos. 5,885,530, 4,981,785, 6,159,750 and 5,358,691.

In an embodiment, a multiplex assay, such as a multiplex ELISA, can be used to detect simultaneously different specific antibodies in a test sample. In embodiments, such methods employ an array, wherein multiple binding agents (for example capture antibodies) specific for multiple antibodies are immobilized on a substrate, such as a membrane, with each capture agent being positioned at a specific, pre-determined, location on the substrate. Methods for performing assays employing such arrays include those described, for example, in U.S. Patent Application Publication Nos. US 2010/0093557A1 and US 2010/0190656A1, the disclosures of which are specifically incorporated by reference herein. If flow cytometry, chemiluminescence, or electron-chemiluminescence technology is employed, multiplex arrays can be used in several different formats. Illustratively, flow cytometric multiplex arrays, also known as bead-based multiplex arrays, include the Cytometric Bead Array (CBA) system from BD Biosciences (Bedford, MA) and multi-analyte profiling (xMAP®) technology from Luminex Corp. (Austin, TX), both of which employ bead sets which are distinguishable by flow cytometry.

In another embodiment, a multiplex ELISA from Quansys Biosciences (Logan, UT) involves coating multiple specific capture reagents at multiple spots (one reagent at one spot) in the same well on a 96-well microtiter plate. Chemiluminescence technology is then used to detect multiple antibodies that bind at the corresponding spots on the plate.

In certain embodiments, a patient can be diagnosed by adding a biological sample (e.g., blood or serum) from a patient to the kit, or components thereof, and detecting the relevant sample antibodies that specifically bind to the Kex peptide reagents. By way of example, the method comprises: (i) collecting blood or serum sample from the subject; (ii) adding subject's sample to the components in the kit, e.g., a holding tube or a substrate; and (iii) detecting the peptide reagents to which the sample antibodies have bound. In this method, the subject's sample, e.g., blood or serum, is brought into contact with the Kex peptide reagent(s). If the anti-Kex peptide antibody(ies) are present in the sample, the antibodies will bind to the Kex peptide reagents, or a subset thereof. In other kit and diagnostic embodiments, blood is not collected from the patient (i.e., it is already collected), and is assayed for the presence of antibodies against the non-naturally occurring Kex peptide. In some embodiments, the antibodies against the non-naturally occurring pan-fungal Kex peptide cross-reacts with one or more, two or more, three or more, or four of the Pneumocystis, Aspergillus, Candida, or Cryptococcus organisms using the kit. Moreover, in other embodiments, the sample may comprise a tissue sample or a clinical sample, which can be processed, e.g., homogenized and/or suspended in medium or buffer, prior to assay. In embodiments, any antibody(ies) found to be present in a test sample from a subject may be isolated, or isolated and purified, and further characterized.

The kit can also comprise a washing solution or instructions for making a washing solution, in which the combination of the capture reagents and the washing solution allows capture of anti-Kex antibodies on the solid support for subsequent detection by, e.g., secondary antibodies, labeled reagent peptides, or mass spectrometry. In a further embodiment, a kit can comprise instructions for suitable operational parameters in the form of a label or separate insert (package insert). For example, the instructions may inform a consumer or user about how to collect the sample, how to wash the anti-Kex peptide antibody and Kex peptide reagent complex after binding has occurred, how to interpret the results, etc. In yet another embodiment, the kit can comprise one or more containers with appropriate positive and negative controls or control samples, to be used as standard(s) for detection, calibration, or normalization.

In another aspect, kits are provided for the treatment or prevention of an infection or disease caused by or associated with two or more of Pneumocystis, Aspergillus, Candida, or Cryptococcus fungal pathogens. In some embodiments, the kit includes an effective amount of a therapeutic or prophylactic antiserum, which contains anti-Kex peptide antibodies or antigen binding fragments thereof that bind/react with a non-naturally occurring Kex peptide. In some embodiments, these antibodies cross react with one or more of Pneumocystis, Aspergillus, Candida, or Cryptococcus, in unit dosage form. In an embodiment, the antiserum is an isolated antiserum. In other embodiments, the kit includes a therapeutic or prophylactic composition containing an effective amount of an anti-fungal immunoprotective agent such as antiserum in unit dosage form. In some embodiments, the kit comprises a device (e.g., nebulizer, metered-dose inhaler) for dispersal of the composition or a sterile container which contains a pharmaceutical composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition is provided together with instructions for administering the pharmaceutical composition containing isolated antiserum to a subject having or at risk of contracting or developing a fungal infection, particularly infection and disease and the symptoms thereof caused by Pneumocystis, Aspergillus, Candida, or Cryptococcus fungal organisms. The instructions will generally include information about the use of the composition for the treatment or prevention of an infection by one or more of Pneumocystis, Aspergillus, Candida, or Cryptococcus fungal organisms. In other embodiments, the instructions include at least one of the following: description of the therapeutic/prophylactic agent; dosage schedule and administration for treatment or prevention of infection or symptoms thereof caused by one or more of Pneumocystis, Aspergillus, Candida, or Cryptococcus fungal organisms; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the presently described embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides such as those described herein, and, as such, may be considered in making and practicing the aspects and embodiments described herein. Particularly useful techniques for particular embodiments will be discussed in the sections that follow.

### EXAMPLES

The following examples are put forth to provide those of ordinary skill in the art with a complete disclosure and description of how to make and use the assay, screening, and therapeutic methods as described herein, and are not intended to limit the scope of the described aspects and embodiments.

### Example 1: Identification of Pan-Fungal Consensus Sequences of Kex1

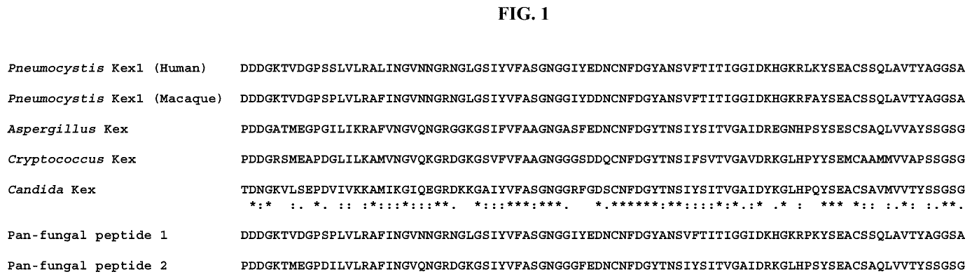

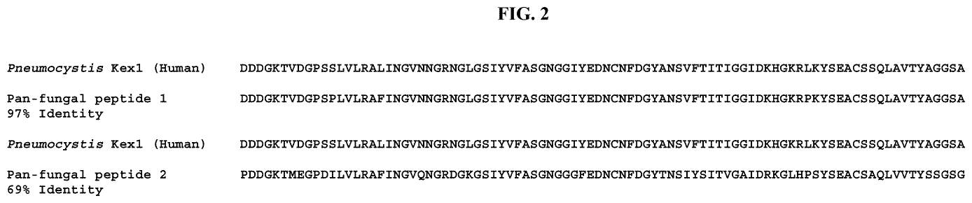

To identify Pan-fungal consensus sequences of Kex1, multisequence alignments of KEX1 peptide sequences from Pneumocystis (Accession No. EU918304.1) (isolated from macaque) Aspergillus fumigatus Kexin (Accession no. XM746441), Candida albicans Kexin (Accession no. AF022372), and Cryptococcus neoformans Kexin (Accession no. XP572303.1) were performed using Clustal Omega (http://www.ebi.ac.uk/Tools/msa/clustalo/) to analyze sequence identity and similarity (FIG. 1). The resulting 90-mer pan-fungal consensus KEX1 peptides (Pan-fungal peptide 1 and Pan-fungal peptide 2) shown in FIG. 2 have approximately 97% and 69% amino acid sequence identities, respectively, with the amino acid sequence of Pneumocystis KEX1 isolated from humans. The corresponding DNA sequence of the Pan-fungal 2 consensus Kex1 peptides were cloned in an E. coli expression vector and the recombinant protein was produced, isolated and purified.

### Example 2: Generation of Recombinant Kexin Proteins

Aspergillus Kex, Cryptococcus Kex, Candida Kex, Pan-fungal Kex peptides 1 and 2 amino acid sequences shown in FIG. 1 were back-translated (www.ebi.ac.uk/Tools/st/emboss_backtranseq/) with an Escherichia coli K-12 codon bias and inserted into the expression vector pET-28b(+) using NcoI and BamHI restriction sites (GenScript). Pneumocystis Kex1 was cloned using the Genbank: EU918304.1 sequence. Each insert contained an additional GC 5′ to the Kex sequences followed by CG to keep the Kex sequence inserts in frame. Plasmids were transformed into Escherichia coli BL21 (DE3) cells and plated on LB agar supplemented with 40 μg/mL kanamycin to select for transformed clones. All recombinant Kexin proteins were expressed and purified as described in Example 3 below.

### Example 3: Procedures for the Purification of Recombinant Kexin Proteins

This example describes a protocol for purifying a recombinantly produced (pET28b vector), (Millipore-Sigma, US), Pneumocystis Kex1, Aspergillus Kex, Cryptococcus Kex, Candida Kex, Pan-fungal Kex peptides 1 and 2 that are histidine tagged. Elution conditions differ for Aspergillus Kex and Cryptococcus Kex proteins due to the insoluble nature of these recombinant proteins.

### Materials and Equipment

A. LB (Lysogeny Broth) growth medium with kanamycin (40 μg/mL), typically in a 1 L volume, pH to 7.5. 10 g NaCl, 5 g Yeast Extract, and 10 g Tryptone Peptone are admixed; the volume is brought to 1 L with distilled/deionized H₂O.
B. 1 M IPTG solution.
C. Extraction buffer: (sterile filtered), 50 mM Sodium Phosphate, 300 mM NaCl, 10 mM imidazole, 6 M Guanidine-HCl, pH 7.4.
D. Wash buffer: (sterile filtered) 50 mM Sodium Phosphate, 300 mM NaCl, 10 mM imidazole pH 7.4.
E. 1 M imidazole solution in 50 mM Sodium Phosphate, 300 mM NaCl, pH 7.4) and 0.2 μm sterile filter (sterile filtered).
F. HisPur™ Cobalt resin (ThermoFisher #89966)
G. Disposable 5 mL polypropylene column (Thermo P #29922).
H. His-tag protease inhibitor cocktail (PIC) (Sigma P #8849).
I. Bradford Dye (Bio Rad P #500-0006).
J. Bovine Serum Albumin Standard (ThermoFisher #23209).
K. Coomassie Blue stain containing 0.2% Coomassie Blue, 7.5% acetic acid and 50% ethanol.
L. Coomasie Blue destain containing 50% methanol, 10% acetic acid and 40% dH₂O.
M. Acrylamide Bis 30% (Sigma P #1001356385)
N. N,N,N′,N′-Tetramethylethylenediamine (TEMED) (Sigma P #1001434505).
O. Sodium dodecyl sulfate (SDS) (10% stock solution).
P. Ammonium persulfate (APS) (10% stock solution).
Q. 1.5 Tris buffer pH 8.8 (187 g Tris Base into 1 L dH₂O, bring pH to 8.8).
R. 0.5 Tris buffer pH 6.5 (60.5 g Tris Base into 1 L dH₂O, bring pH to 6.5).
S. Spectra™ Multicolor Ladder-Broad range stained (Thermo P #22634).
T. SDS-PAGE sample buffer (4×).
U. Hoefer gel casting system (model SE250).

The procedure used is as follows:
A. Culture and induce protein expression in E. coli:
   a. Streak Kexin construct on pET28b(+) onto LB KAN agar plate and incubate at 37° C. overnight (O/N) or at RT on the bench top until adequate bacterial growth/colonization is obtained. (Plates can be stored at 4° C. for ~1 month)
   b. Inoculate a single colony into 10 mL liquid LB KAN 40 (10 μL of 40 mg/mL KAN per 10 mL LB), (allowing ~1:5 liquid to air ratio), and grow at 37° C. overnight with shaking.
   c. Following overnight incubation, dilute culture 1:50 into liquid LB KAN 40 (4 mL of overnight culture into 196 mL of fresh medium) and leave at 37° C. on shaker.
   d. Grow cultures to an OD₆₀₀ 0.5 and then add 1 mM IPTG to induce expression; leave at 37° C. on shaker for 4-5 hours.
   e. Split total volume of culture among five 50 mL Oakridge tubes (~40 mL culture per tube). For scaling up: can use 250 mL Oakridge tubes for larger volumes.
   f. Harvest cells by centrifugation at 6,000×g and 4° C. for 25 minutes (Can use SS-34 or SLA-1500 rotor).
   g. Pour off supernatant and freeze cell pellets at −80° C. until time of use. Do not store E. coli pellets for longer than two weeks prior to protein extraction.
B. Protein purification using Talon metal affinity resin:
   a. Thaw pellet on ice and re-suspend cell pellet in 10 mL extraction buffer+200 μL PIC.
   b. Incubate at 4° C. for 2 hours minutes on nutator.
   c. Centrifuge suspension at 10,000×g and 4° C. for 20 minutes (use SS-34 rotor).
   d. Collect supernatant and keep on ice until Talon resin is prepared.
   e. Prepare polypropylene elution column by suspending column in the upright position; adding a few drops of wash buffer to a porous disc, then using reverse end of a Pasteur pipette to depress disc evenly to the bottom of the column.
   f. Prepare Talon resin: Resuspend Talon resin by gently shaking and add 3.5 mL of resin to a 15-mL conical tube and spin for 5 minutes at 500×g. Carefully remove ethanol layer without disturbing resin. Add 10 mL of deionized water to wash resin and spin again for 5 minutes at 500×g. Remove supernatant carefully and discard. Equilibrate resin in 10 mL of extraction buffer and spin for 5 minutes at 500×g. Remove supernatant carefully and discard.
   g. Batch bind clarified lysate and equilibrated resin by mixing together and nutating for 1 hour at 4° C.
   h. Add lysate and resin suspension to the prepared polypropylene elution column. Discard flow through.
   i. Wash resin with 15 column volumes of extraction buffer followed by 15 column volumes of wash buffer.
   j. Elution of Pneumocystis Kex1, Candida Kex, and Pan-fungal peptide 2.
   Elute in 1.5 mL fractions with increasing imidazole concentration in wash buffer and collect elution fractions.
      Elution gradients
      a. Add 1.5 mL of 75 mM imidazole in wash buffer and collect fraction.
      b. Add 1.5 mL of 100 mM imidazole in wash buffer and collect fraction.
      c. Add 1.5 mL of 125 mM imidazole in wash buffer and collect fraction.
      d. Add 1.5 mL of 150 mM imidazole in wash buffer and collect fraction.
      e. Add 1.5 mL (×2) of 175 mM imidazole in wash buffer and collect fraction.
      f. Add 1.5 mL (×3) of 200 mM imidazole in wash buffer and collect fraction.
      j. Elution of Pneumocystis Kex1, Pan-fungal peptide 2.
   k. Elution of Aspergillus Kex and Cryptococcus Kex (Insoluble proteins)
      a. Resuspend resin in 1.5 mL of 1% SDS and transfer into two 1.5 mL tubes.
      b. Boil suspension in a heating block 95° C.
      c. Centrifuge suspension for 5 minutes at 500×g.
      d. Collect supernatant fraction containing denatured protein.
      e. Repeat steps a-d three times to recover additional protein.
   l. Add 20 μL PIC to each fraction of interest and store at 4° C. (Imidazole solutions should be stored on ice prior to use.)
C. Quantification of protein in elution fractions (Bradford Assay-low concentration standard curve):
   a. Remove BSA-100 μg/mL from freezer (4° C.) and thaw on ice.
   b. Set up cuvettes for standard curve and add the specified amounts of both the thawed BSA/dH₂O from the below table (Table 1).

TABLE 1

| Final Conc. | dH₂O | BSA-100 μg/mL |
|---|---|---|
| (μg/mL) | (μL) | (μL) |
| 0 | 200 | 0 |
| 0.25 | 197.5 | 2.5 |
| 0.5 | 195 | 5 |
| 1 | 190 | 10 |
| 2 | 180 | 20 |
| 4 | 160 | 40 |
| 8 | 120 | 80 |

c. Add an additional cuvette for each fraction and dilute samples 1:50 (20 μL sample+180 μL dH₂O).
d. Prepare Bradford dye 1:4 in dH₂O (10 mL dye+30 mL dH₂O) and add 800 μL to each cuvette (final volume 1 mL).
e. Mix cuvettes individually via inversion and incubate at RT for 15 minutes.
f. After incubation, add 200 μL of the 0 μg/mL BSA standard in replicate to wells A1 and A2 of 96 well flat bottom plate followed by the addition 0.25 μg/mL BSA standard to B1 and B2. Continue to add the BSA standard in increasing concentration to the plate in the same order.
g. Once the entirety of the BSA standard is added to the plate, load samples in replicate into the wells immediately below until no rows remain and then proceed to the top row of the next two columns.
h. After all samples are loaded onto the plate read at 595 nm—“Low-conc. Std. Curve.”
i. Record the linear regression (R²) and BSA standard curve values (Data obtained from assays with R²<0.95 should not be used). Raw values for samples represent a 1:50 dilution and should therefore be multiplied by 50 in order to convert back into μg/mL. Once the concentrations of protein have been determined, fractions intended for plate coating (e.g., ELISA/ELISPOT), injection, etc. must be run on a 15% 2 mm SDS-PAGE gel to evaluate purity.
D. Identification of protein via SDS-PAGE gel Coomassie Blue staining:
   a. For each gel, wash 1× glass cover plate, 1× white aluminum backing plate, 2× black plate spacers, and 1× white 10 lane stacking comb with dH₂O. Rinse with 70% ethanol solution. Confirm that all solidified gel residue from previous use is removed before casting.
   b. After all materials have air-dried, take the backing plate and lay it flat on the bench top and place a spacer on each side of the plate before sandwiching with the clear glass cover plate. Confirm that the notches of the spacer are properly aligned to the edges of both plates.
   c. Loosen all screws on the casting block and slide the sandwiched plates with spacers into the caster. Confirm that all plates and spacer are even and aligned. Leave ~3 mm of the sandwiched plates protruding from the bottom of the casting block before carefully tightening the screws so as not to crack the plates.
   d. Place the casting block into the holder and set the black plastic plugs into the holder. Turn plugs to depress casting block into the black rubber mat of the holder. Confirm that the bottom of the plates is well sealed by the rubber of the holder in order to avoid leaks.
   e. Prepare separating/running gel according to the recipe below for a 15%-SDS PAGE gel and add solution to the cavity between aluminum backing plate and glass cover plate. Allow ~1.5-2 cm of space at the top of the sandwiched plates for stacking gel. Add ~1 mL of dH₂O to casting block. The gel will begin to polymerize once the APS/TEMED are added to the solution.

| Separating Gel | 15% | |
|---|---|---|
| Acrylamide Bis 30% | 5 | mL |
| Water (dH₂O) | 2.34 | mL |
| 1.5 Tris Buffer pH 8.8 | 2.5 | mL |
| SDS (10%) | 100 | μL |
| APS (10%) | 50 | μL |
| TEMED | 10 | μL |

f. Once the gel has hardened (approximately 35 minutes), remove the layer of water and prepare the stacking gel solution from the recipe below. Add solution quickly.

| Stacking Gel | 15% |
|---|---|
| Acrylamide Bis 30% | 700 μL |
| Water (dH₂O) | 3.2 μL |
| 0.5 Tris Buffer pH 6.5 | 1250 μL |
| SDS (10%) | 50 μL |
| APS (10%) | 60 μL |
| TEMED | 20 μL |

g. Immediately place the white 10 lane stacking comb into the stacking gel and allow to fully polymerize (about 10 minutes).
h. Prepare samples to run on gel: (5 μg protein per well)
   i. If the fraction concentration is <165 μg/mL, use 22.5 μL of sample+7.5 μL 4× Sample Buffer;
   ii. If the fraction concentration is >165 μg/mL=>sample vol.=5 μg/(conc. (μg/mL)/1000) & 4× Sample Buffer vol.=(⅓)*sample vol.
i. Heat-inactivate all samples for 10 minutes at 56° C. in the water bath.
j. Remove 15% SDS-PAGE gel from casting block and attach to the running apparatus with 2× red clips. Fill the cavity of the running apparatus and the bottom tray with 1×SDS-PAGE running buffer. The stacking comb can now be removed. Add 10 μL of the Broad Range stained (P #26634) SPECTRA™ Multicolor Ladder to first well of the gel followed by 30 μL of the prepared samples to the subsequent wells.
k. Once all samples are loaded, attach the electrodes to their appropriate terminals and turn on the power supply (red to red, black to black). Allow the gel to run at ~80-120 volts for 1.5-2.5 hours until the dye band runs of the bottom of the gel. At that point turn off the machine and disconnect the electrodes (Note: Lower voltages and lower time intervals increase the quality of the resulting gel).
l. Drain the running buffer from the running apparatus. Remove the red clips, spacers, and gently detach the glass cover plate from the gel casting frame. Use the hard plastic straight edge of the gel scraper to cleave the stacking gel off and into the trash. Divide gel as necessary for further assays, i.e. Western Blot, etc. (it is not necessary to notch a corner of the gel to establish orientation because of the stained ladder used).
m. For the separating gel that will be stained, wash 3× w/ dH₂O for 15 minutes. Add ~25 mL of Coomassie Blue stain to the gel for 2+ hours or overnight if necessary. Destain with Coomassie Blue de-stain until optimal band color/gel transparency is obtained. Take a picture and save as JPG/TIF file.

### Example 4: Generation of Immune Sera

Immunization of Rhesus macaque with Recombinant Pan-Fungal 2 Peptide

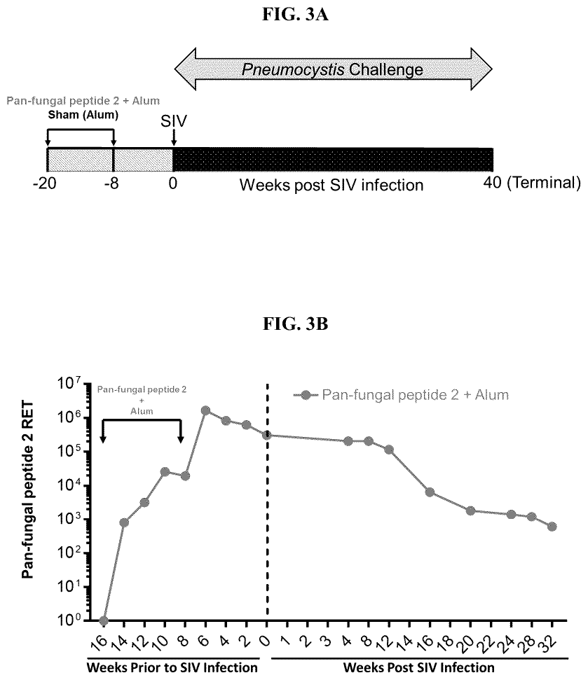

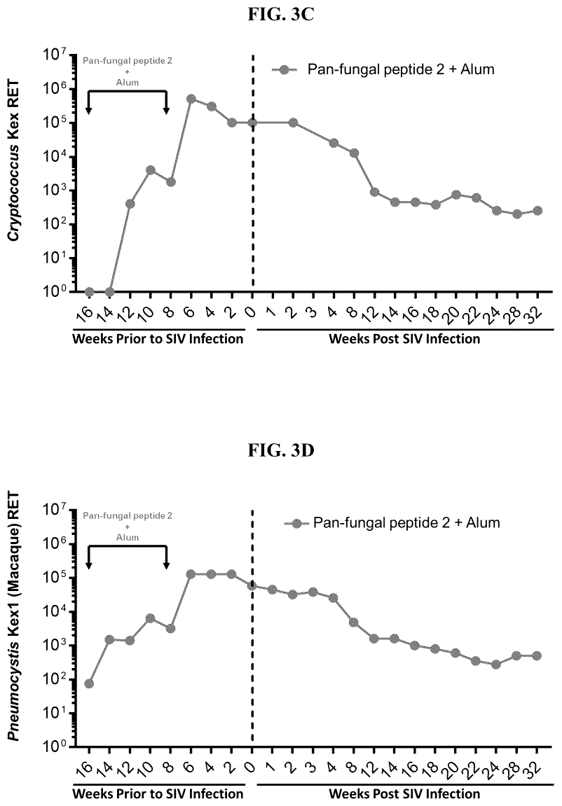

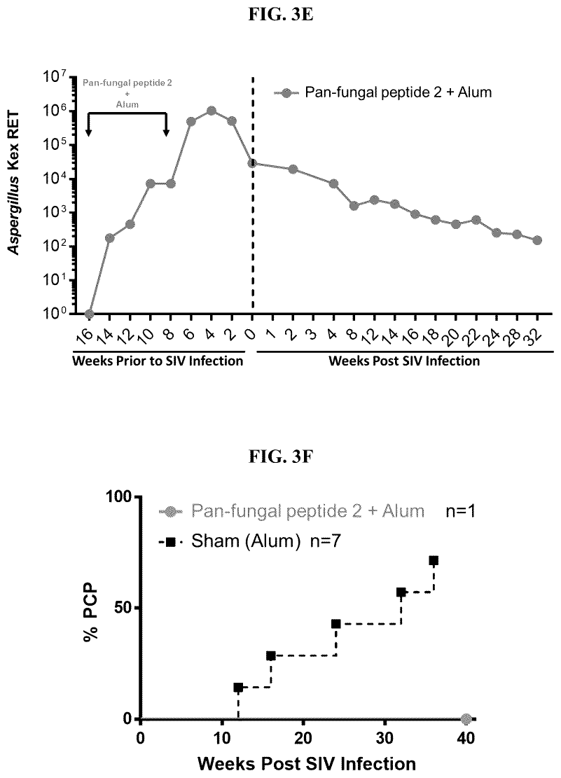

### (Study Design FIG. 3A)

An adult Rhesus macaque (monkey subject number 2116) was intramuscularly immunized with 100 μg of Pan-fungal peptide 2. The monkey received a booster at 8 weeks post administration and was infected with SIV 8 weeks post boost. Referring to FIGS. 3B-3E, ELISA analysis of serum samples collected from monkey subject number 2116 was used to determine the reciprocal endpoint titers (RET) with Pan-fungal peptide 2, Cryptococcus Kex, Pneumocystis Kex1, and Aspergillus Kex as targets. As used herein, RET is the reciprocal of the highest analyte (e.g., antibody or antiserum) dilution that gives a reading above the cutoff value. For the RET analyses, serum obtained from the test animal is assayed to determine the presence of immunoglobulin (IgG) that is directed to, binds, and/or has activity against the target protein(s). The peak titers for each target was greater than 10⁵, which demonstrates the presence of primary antibodies that recognize kexin peptides from multiple fungal species. As illustrated in FIG. 3F, more than half of the of the monkeys receiving sham vaccination developed Pneumocystis pneumonia, while the monkey receiving the Pan-fungal vaccination did not.

### Example 5: Kex Peptide Enzyme Linked Immunosorbent Assay (ELISA)

This example describes a protocol for performing an ELISA immunoassay utilizing the Pneumocystis Kex1 protein, the Pan-fungal peptide 2, or a Kex peptide derived from one or more of Pneumocystis, Aspergillus, Candida, or Cryptococcus. The ELISA is conducted to detect (and quantify) the presence of anti-fungal Kex peptide antibodies in a sample, e.g., blood, plasma, serum, bronchoalveolar lavage, or biological fluid sample. The anti-Kex peptide antibodies to be detected (and quantified) can be directed against, reactive with and/or bind to the Pan-fungal peptide 2 or to the Kex peptide of one or more of the Pneumocystis, Aspergillus, Candida, or Cryptococcus Kex peptides.

### Materials and Equipment

A. KEX1 protein, which may be purified as described in Example 2
B. 1×PBS
C. Immulon high-binding (4HBX) Flat bottom microtiter plates (Thermo #3855)
D. Blocking buffer: 5% skim milk in 1×PBS
E. Wash buffer: 1× Phosphate-buffered Saline (PBS)+0.05% Tween-20
F. Secondary Antibody: Goat anti-human immunoglobulin-conjugated horseradish peroxidase (1:10,000 for IgG; Sigma-Aldrich).
G. Normal human plasma (Atlanta Biologicals, Inc., Lawrenceville, GA). Negative/normal control plasma with undetectable absorbance at OD₄₅₀ (i.e., equal to or less than dilution buffer alone) in KEX-ELISA at a dilution of 1:100 is used as negative controls.
H. Substrate: 3,3′,5,5′-Tetramethylbenzidine (TMB) peroxidase substrate (such as SureBlue TMB substrate, 1-component; KPL, Inc.)
I. Stop solution: 1M H₂SO₄
J. Adhesive sealing film for microplates (Plate sealers) (such as SealPlate non-sterile films from Excel Scientific, cat #100-SEAL-PLT)
K. 96-well plate reader (any system capable of reading OD at a wavelength of 450 nm).

The procedure for performing the ELISA is as follows:
A. Coating/blocking ELISA plates with Kex1 protein:
   a. Prepare mkKex protein in 1×PBS at 5 μg/mL. Add 50 μL of diluted KEX1 per well of Immulon 4HBX flat-bottom ELISA plates. Cover plates tightly with Parafilm or plate sealers and incubate overnight at 4° C.
   b. Following overnight incubation, remove buffer by flicking into sink or bucket and tap plate onto absorbent pad or paper towels to remove excess. Wash plates 2× with wash buffer (PBS 0.05% Tween-20) (approximately 200 μL wash buffer per well for each wash, flicking and tapping plate between washes).
   c. Add 100 μL of blocking buffer (5% milk/PBS) to each well and incubate for 1 hour at 37° C.
   d. Empty plates, wash 2× with wash buffer. The plates can be sealed and frozen at −20° C. at this step, until ready for use.
B. Handling of plasma or other infectious fluids (e.g., bronchoalveolar lavage (BAL) fluid supernatant, etc.)—First-time use.
   a. Remove plasma aliquot from −80° C. freezer.
   b. Option 1: Heat-inactivate entire aliquot at 56° C. for 30 minutes.
      Option 2: If heat inactivation of the plasma sample would be detrimental to other potential uses, thaw sample at 4° C. or on ice. Remove an aliquot (~100 μL), transfer to a new tube, and heat inactivate (30 minutes, 56° C.). Return the remaining sample to the −80° C. freezer, noting that it has been thawed 1×.
   c. Centrifuge sample at >10,000×g for 1-2 minutes to pellet aggregates prior to use.
   d. To prevent contamination in storage, add ~0.01 to 0.02% NaN₃. Store sample aliquot for up to 6 months at 4° C. For subsequent assays, no further heat inactivation is needed; however, the sample should be centrifuged briefly prior to each use.
C. ELISA for endpoint titer determination (plasma):
   a. Dilute plasma 1:100 in blocking buffer. Add 50 μL of diluted plasma and make serial 2× (or 4×, if needed) dilutions directly in the plate (final volume in each well should be 50 μL) for generation of endpoint titers. Perform assay in duplicate; set up enough plates for all isotypes of interest, e.g., if there are 10 samples and endpoint titers are to be generated for both IgG and IgM-KEX1 antibodies, this would require setting up 4 plates (duplicate plates for both IgG and IgM). Include a negative/normal control on each plate. Cover plates with plate sealers and incubate overnight at 4° C.
   b. Empty plate (flicking and tapping), wash 4× w/ wash buffer.
   c. Add 50 μL of secondary antibody (diluted in block) to each well (see appropriate dilutions under Materials and Equipment above). Incubate 1 hour at 37° C.
      d. Empty the plate and wash 6× with wash buffer.
      e. Add 100 μL of TMB to each well, protect from light and incubate for 30 minutes at 37° C.
      f. Add 25-50 μL of stop solution (1M H₂SO₄) to each well.
      g. Read OD of plates (on any standard plate reader) at 450 nm within 20 minutes of adding stop solution.

The majority of healthy adults (both humans and non-human primates) have circulating antibodies to Pneumocystis; therefore, when selecting a control sample to be used for calculating endpoint titers, plasma samples must be screened from healthy donors to determine and obtain an appropriate control. In plasma from an appropriate normal/negative control, the KEX1 OD₄₅₀ at a 1:100 dilution should be not more than 0.1; however, the lower the OD of the normal/negative control plasma, the better the control is. To control for plate-to-plate variability, the same normal/negative control should be used on all plates following the selection of an appropriate normal/negative control.
D. ELISA for endpoint titer determination (BAL Supernatant).
   a. Dilute BAL supernatant 1:100 in normal saline.
      i. Determine the urea concentration of the BAL supernatant and corresponding plasma sample using QuantiChrom Urea assay (BioAssay Systems Cat #DIUR-500).
         1. Follow instructions on kit insert, diluting plasma 1:10 in distilled water and using BAL supernatant without dilution.
         2. Plate plasma samples in the wells of a 96-well plate adding 5 μL of standard (1:10 dilution), blank (distilled water) and sample (1:10 dilution) in duplicates.
         3. Plate BAL supernatant in the wells of a 96-well plate adding 50 μL of standard (diluted to 5 mg/dL), blank (distilled water) and sample (undiluted).
         4. Add 200 μL working reagent (included in kit) and tap lightly to mix.
         5. Incubate plasma plate for 20 minutes at room temperature (RT) and read at OD₅₂₀ on a spectrophotometer.
         6. Incubate BAL supernatant plate for 50 minutes at RT and read at OD₄₃₀.
         7. Calculate urea concentrations ([urea]) for plasma and BAL supernatant as follows: [urea]=(ODsample−ODblank)/(ODstandard−ODblank)*[standard]. The concentration of standard for plasma will be 50 mg/dL and will be 5 mg/dL for the BAL supernatant.
         8. Calculate 1:100 dilution of BAL supernatant as follows:
         a. Find the 1:100 dilution factor of bal to plasma
         i. Dilution factor=100/(plasma [urea]/bal [urea])
         b. Calculate volumes for dilution for 500 ul total sample
         i. Volume of sample=500 μL/dilution factor
         ii. Volume of saline=500 μL−volume of sample
         9. Add the volume of sample and volume of saline to make 1:100 diluted BAL supernatant sample.
   b. Add 50 μL of diluted BAL supernatant and make serial 2× (or 4×, if needed) dilutions directly in the plate with normal saline (final volume in each well should be 50 μL) for generation of endpoint titers. Perform assay in duplicate; set up enough plates for all isotypes of interest, e.g., if there are 10 samples and endpoint titers are to be generated for both IgG and IgM-KEX1 antibodies, this would require setting up 4 plates (duplicate plates for both IgG and IgM). Include a negative/normal control in each plate, as described above. Cover plates with plate sealers and incubate overnight at 4° C.
   c. Empty plate (flicking and tapping), wash 4× w/ wash buffer.
   d. Add 50 μL of secondary antibody (diluted in block, see appropriate dilutions under Materials and Equipment) to each well. Incubate for 1 hour at 37° C.
   e. Empty the plate and wash 6× with wash buffer.
   f. Add 100 μL of TMB to each well, protect from light and incubate for 30 minutes at 37° C.
   g. Add 25-50 μL of stop solution (1M H₂SO₄) to each well.
   h. Read OD of plates (on any standard plate reader) at 450 nm within 20 minutes of adding stop solution.
E. Determining Endpoint Titers.
   a. Plot OD readings from each sample (at all dilutions) in Excel, or similar program, as a line graph. For the normal/negative control sample, add 0.025 to each value prior to plotting as described below.
   b. The endpoint titer is defined by the dilution at which the test sample gives the same OD reading as that of the negative control (i.e., where the lines meet). Generally, the reciprocal endpoint titer is reported; thus, if the dilution is 1:1600, the endpoint titer is reported as 1600.
   c. Calculate endpoint titers from each of the duplicate plates, to confirm that the results are consistent between plates. Acceptable error is within one dilution. If reciprocal endpoint titers (RET) from duplicate plates fall within one dilution, average the titers (e.g., when doubling-dilutions are made, and a sample from plate 1 has a RET of 1600 and the RET from plate 2 is 3200, then the average titer is 2400). If endpoint titers on duplicate plates do not fall within one dilution of each other, repeat the ELISA on one additional plate and average the 2 values which are closest.

### Example 6: Pan-Fungal Vaccination

An adult Rhesus macaque was intramuscularly immunized with 100 μg of pan-fungal 2 peptide and aluminum hydroxide (Imject Alum, ThermoScientific) mixed in a 1:1 ratio and then boosted 8 weeks later with 50 μg of the pan-fungal 2 peptide and aluminum hydroxide. At eight weeks post-boost, the monkeys were infected with simian immunodeficiency virus (SIV) and challenged with Pneumocystis (FIG. 3A). Seven additional Rhesus macaques were vaccinated with a sham vaccine, which consisted of the aluminum hydroxide but did not contain any Kex peptide. Referring to FIG. 3F, the percentage of monkeys diagnosed with Pneumocystis pneumonia increased steadily from week 10 to week 40 for those monkeys that received a sham vaccination. The monkey vaccinated with Pan-fungal 2 peptide did not develop pneumonia.

### Example 7: Wild-Type Pneumocystis Kex1 Vaccination

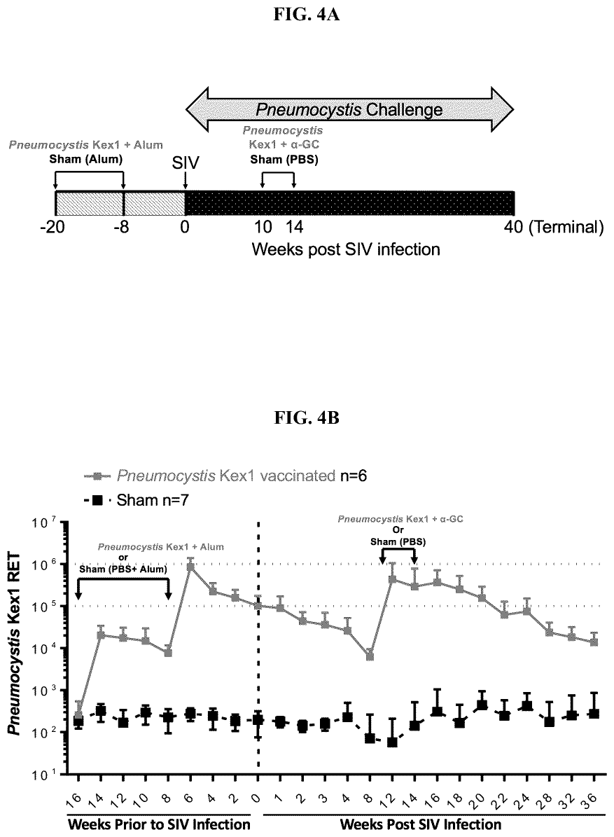

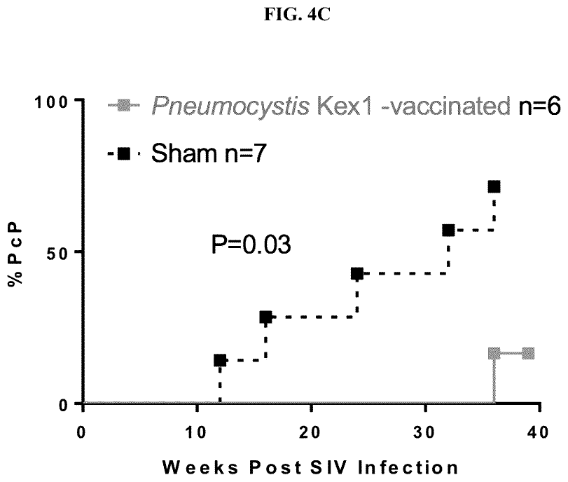

Adult Rhesus macaques were intramuscularly immunized with 100 μg Pneumocystis Kex1 (wildtype Kex1: DDDGKTVDGPSPLVLRAFINGVNNGRNGLGSIYVFASGNGGIY DDNCNFDGYANSVFTITIGGIDKHGKRFAYSEACSSQLAVTYAGGSA), (SEQ ID NO: 3), and aluminum hydroxide (Imject Alum, Thermo Scientific) mixed in a 1:1 ratio and then boosted 8 weeks later with 50 μg wildtype Kex1 and aluminum hydroxide. At eight weeks post-boost, the monkeys were infected with simian immunodeficiency virus (SIV) and challenged with Pneumocystis. At 10 and 14 weeks post infection, the monkeys were boosted with 100 μg wildtype Kex1 and 5 μg α-GC. Seven additional adult Rhesus macaques were intramuscularly immunized with alum mixed in a 1:1 ratio and then boosted 8 weeks later with alum. At eight weeks post-boost, the monkeys were infected with simian immunodeficiency virus (SIV) and challenged with Pneumocystis (FIG. 4A) and boosted at 10 and 14 weeks post infection with PBS. Referring to FIG. 4B, ELISA analysis of sera samples collected from Pneumocystis Kex1-vaccinated in sham controls. The peak titers for the wild type Kex1 vaccinated group was greater than 10⁵ following both the second wildtype Kex1 with alum and wildtype Kex1 with α-GC boosts. Referring to FIG. 4C, only one of the six wildtype Pneumocystis Kex1-vaccinated monkeys developed Pneumocystis pneumonia, and this occurred at the end of the 40-week Pneumocystis challenge.

### Example 8: Therapeutic Vaccination in SIV-Immunocompromised Nonhuman Primates (NHPs)

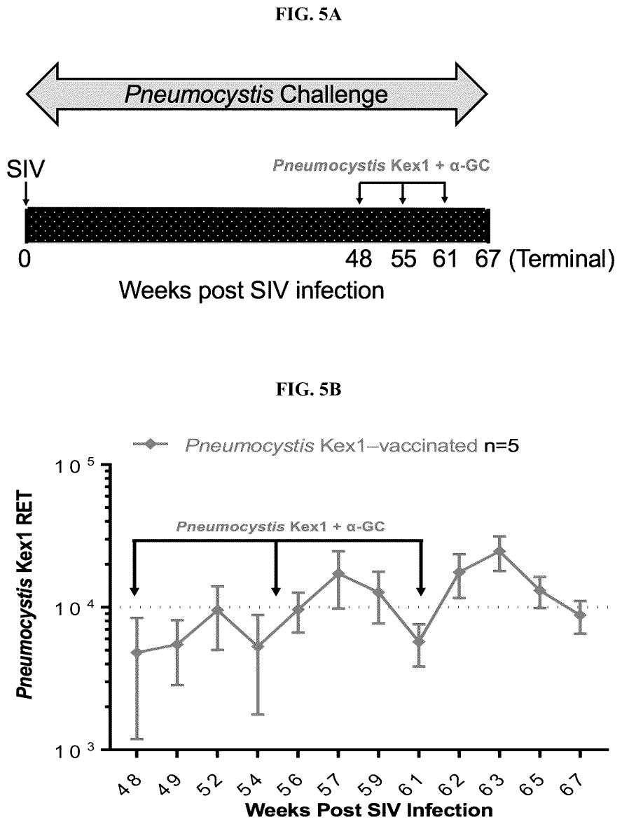

Adult Rhesus macaques were SIV infected and boosted 48, 55, and 61 weeks later with 50 μg Pneumocystis Kex 1 and 5 μg alpha-galactosylceramide (αGC) as an adjuvant (FIG. 5A). Referring to FIG. 5B, ELISA analysis of sera samples collected from Pneumocystis Kex1-vaccinated animals. The peak reciprocal endpoint titers for the wild type Kex1 vaccinated group were greater than 10⁴ following second and third boost with α-GC at 57 and 63 weeks post-infection.

### Example 9: Evaluation of the Immunogenicity and Protective Efficacy of PF,KEX1 in a Murine Model of Aspergillus fumigatus (AF) Infection

A mouse model of invasive pulmonary aspergillosis (IPA) was used to assess whether mice immunized with the panfungal kex peptide (Pan-fungal peptide 2) and adjuvant would mount a humoral immune response that protected the immunized animals from subsequent infection by Aspergillus pathogen as described below.

### Humoral Immune Responses to Pan-Fungal Peptide 2

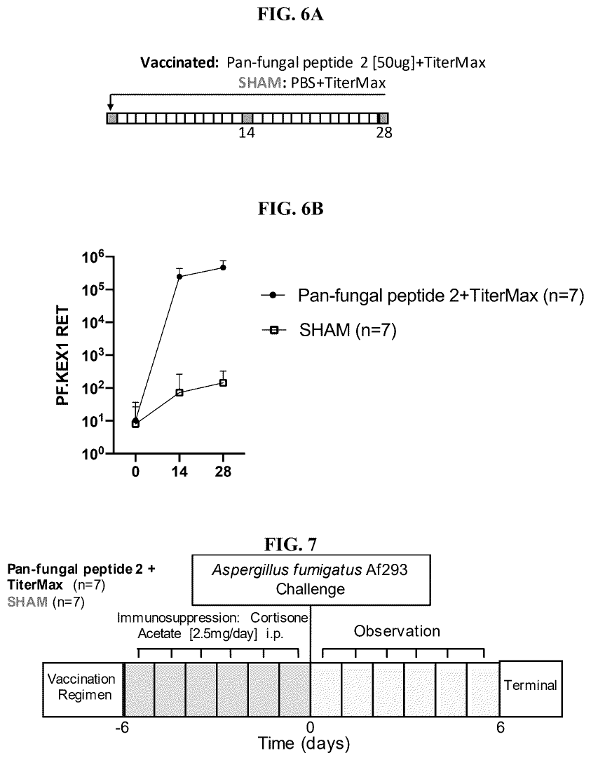

The immunogenicity of the Pan-fungal peptide 2immunogen (termed “vaccine candidate” herein) was evaluated by immunization (vaccination) of animals with recombinant Pan-fungal peptide 2 and TiterMax adjuvant or with PBS and TiterMax, according to the schedule shown in FIG. 6A. FIG. 6B illustrates that anti-Pan-fungal peptide 2 antibody titers significantly increased following immunization (vaccination) of the mice and peaked at 28 days post immunization (mean=5×10⁵) compared with the sham-immunized cohort (**p 0.002).

### Pan-Fungal Peptide 2 Immunization Protects Against Invasive Pulmonary Aspergillosis (IPA) in Immunosuppressed CF-1 Mice

Following vaccination, seven (7) Pan-fungal peptide 2-immunized mice and 7 sham-immunized mice were immunosuppressed for six days before they were challenged intranasally with 5×10⁶ Aspergillus fumigatus (Af293) conidia (FIG. 7). The mice were then observed twice daily for ruffled fur, labored breathing, weight loss and drops in body temperature, which are signs of aspergillosis. Over the observation period, 3 of the sham-immunized cohort developed evidence of aspergillosis and met the criteria for euthanasia. None of the Pan-fungal peptide 2-immunized mice exhibited severe symptoms or succumbed to aspergillosis during the course of the experiment. Survival of the Pan-fungal peptide 2-immunized mice was significantly higher compared to that of the sham-immunized mice (*p=0.05). In addition, the sham-immunized mice had 1.7 times the relative risk of invasive pulmonary aspergillosis (IPA)-related mortality compared to the risk found in Pan-fungal peptide 2-immunized animals (Table 2).

TABLE 2
Association between Pan-fungal peptide 2 Vaccination
and IPA-related mortality

| | Pan-fungal peptide | |
|---|---|---|
| Cohort | 2-Vaccinated | Sham-Vaccinated |
| All animals | n = 7 | n = 7 |

Primary Outcome

| | | |
|---|---|---|
| IPA-related mortality | 0 | 3 |
| No IPA-related mortality | 7 | 4 |

Relative Risk

| | | |
|---|---|---|
| Value | — | 1.750 |
| 95% CI, Koopman | — | 0.9974-2.981 |
| asymptotic score | | |

Association between Pan-fungal Peptide 2 Vaccination
and IPA-related mortality

| | | |
|---|---|---|
| Fisher's exact test p value | — | *0.05 |

CI, confidence interval;
Invasive Pulmonary Aspergillosis (IPA);
*p < 0.05

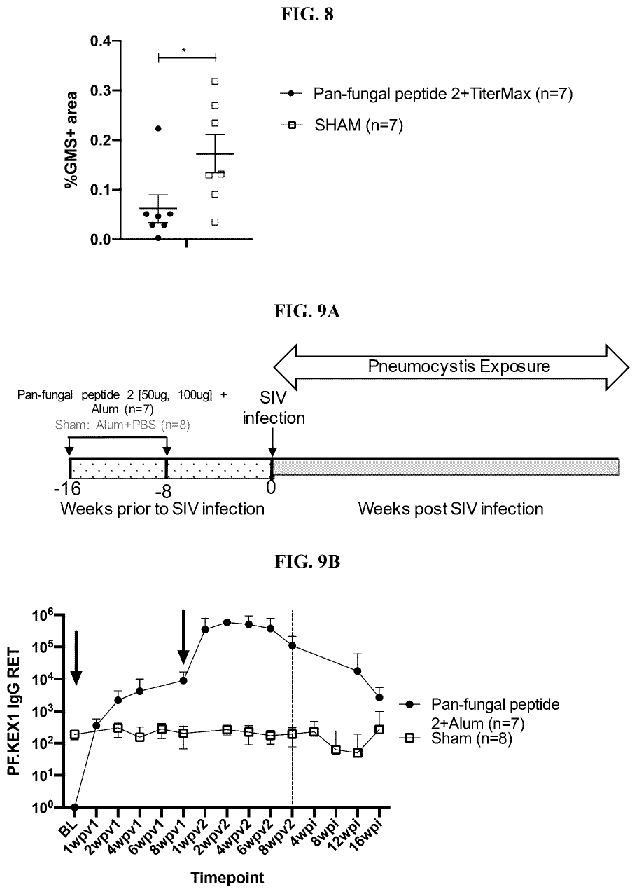

Table 2 presents the association and relative risk analysis comparing vaccination to observation of humane endpoint criteria. Pan-fungal peptide 2 vaccination showed a significant association with IPA-related mortality (*p=0.05), while sham-vaccinated mice had 1.75 times the relative risk of IPA-related mortality. These results were further supported by an analysis of fungal burden, which was conducted using GMS staining histological quantification. A. fumigatus pulmonary fungal burden was significantly reduced in Pan-fungal peptide 2-immunized mice compared to that of the sham-immunized cohort (FIG. 8), (*p=0.026).

Materials and Methods used in this Example are as follows:

### Study Design

Fourteen (14) CF-1 mice (Charles River Laboratories) were randomly assigned to immunization and sham cohorts. Blood was collected at baseline (day 0), as well as at 14 and 28 days following immunization (FIG. 6A). Plasma samples were stored at −80° C.

### Vaccine Construction and Purification

A 90-amino recombinant protein, Pan-fungal peptide 2 (SEQ ID NO: 2) was cloned into the pET28b(+) expression vector (Novagen) in Escherichia coli BL21(DE3) pLysS (ThermoFisher Scientific) and purified by affinity chromatography.

### Immunization and Immunosuppression

Seven (7) mice were immunized subcutaneously at the base of the tail with 50 μg Pan-fungal peptide 2 prepared 1:1 with TiterMax adjuvant according the adjuvant guidelines. An additional seven (7) mice were sham-immunized with PBS and TiterMax. 28 days following immunization, all mice began an immunosuppressive regimen of 2.5 mg cortisone acetate per mouse in PBS with 0.5% methylcellulose and 0.01% Tween-80, injected subcutaneously. This regimen was administered for six days (FIG. 7), during which time trimethoprim sulfamethoxazole was added to the drinking water to control secondary infections.

Aspergillus fumigatus Challenge and Monitoring

A. fumigatus Af293 conidia were maintained on solid 1% glucose minimal medium for 72 hours, harvested in 0.010% Tween-20, counted with a hemocytometer, and then were diluted in PBS. Mice were inoculated with 5×10⁶ conidia in 40 dl PBS via intranasal inoculation following the six days of cortisone acetate immunosuppression as described above.

Following challenge, mice were monitored twice daily for changes in weight, temperature, and appearance. If weight loss exceeded 20 percent of the baseline body weight or body temperature fell below 29° C. in addition to exhibiting ruffled fur and labored breathing, the animals were humanely sacrificed. At six days following challenge, all remaining animals were humanely sacrificed and their lungs were collected for analysis.

### Fungal Burden

Following sacrifice, the right lungs of all animals were stored in 10% neutral buffered formalin. The fixed lung tissue was embedded in paraffin, cut, and stained with Gomori's modified methanamine silver stain. Images of five distinct fields were taken and fungal burden was quantified according to the guidelines provided by Stolz et al., 2018, J. Vis. Exp., (133)e57155:1-8.

### Statistical Analysis

All statistical analyses were performed using GraphPad Prism (GraphPad Software, La Jolla, CA). Reduction in IPA-related mortality was assessed using Fisher's exact test. The relative risk in IPA-mortality was also determined using Koopman asymptotic score. Differences in fungal burden by GMS staining were analyzed by Mann-Whitney U tests.

### Example 10: Evaluation of the Immunogenicity and Protective Efficacy of Pan-Fungal Peptide 2 in a Non-Human Primate Model of Pneumocystis and HIV(SIV) Co-Infection

### Humoral Immune Responses to Pan-Fungal Peptide 2

The immunogenicity of the Pan-fungal peptide 2 immunogen (called “vaccine candidate” herein) was evaluated by immunization (vaccination) of non-human primates with recombinant Pan-fungal peptide 2 and alum or with PBS and alum, according to the schedule shown in FIG. 9A. Anti-Pan-fungal peptide 2 antibody titers significantly increased in animals following immunization and peaked at two weeks post boost (mean=4×10⁵), while no significant change in antibody titers was observed in the sham-immunized cohorts (FIG. 9B).

### Pan-Fungal Peptide 2Immunization Protects Against Pneumocystis Pneumonia

Following the vaccination regimen illustrated in FIG. 9A, all animals were infected with Simian Immunodeficiency Virus (SIV) and were monitored monthly for Pneumocystis. Over the course of SIV infection, 5 macaques in the sham-immunized cohort tested positive for Pneumocystis pneumonia (PCP) by PCR and bronchoalveolar lavage (BAL) smear by 32 weeks post SIV infection. None of the Pan-fungal peptide 2-immunized macaques exhibited symptoms or tested positive for PCP by this same timepoint. This finding demonstrates a significant reduction in the number of PCP cases in Pan-fungal peptide 2-immunized animals (p 0.035). Table 3 below presents data related to the association between Pan-fungal peptide 2 immunization (vaccination) and Pneumocystis pneumonia (PCP) disease in study animals, as well as a significant association between Pan-fungal peptide 2 immunization (vaccination) and PCP (p 0.026). The association and relative risk of immunization of animals with Pan-fungal peptide 2 and the diagnosis of PCP is shown in Table 3. Furthermore, the relative risk of a sham-immunized (vaccinated) animal acquiring PCP was determined to be 3.3 times that of a Pan-fungal peptide 2-immunized (vaccinated) animal (Table 3).

TABLE 3
Association between Pan-fungal peptide 2
Vaccination and Pneumocystis pneumonia

| | Pan-fungal peptide | |
|---|---|---|
| Cohort | 2-Vaccinated | Sham-Vaccinated |
| All animals | n = 7 | n = 8 |

Primary Outcome

| | | |
|---|---|---|
| PCP | 0 | 5 |
| No PCP | 7 | 3 |

Relative Risk

| | | |
|---|---|---|
| Value | — | 3.333 |
| 95% CI, Koopman asymptotic score | — | 1.409-4.088 |

Association between Pan-fungal peptide 2
Vaccination and PCP

| | | |
|---|---|---|
| Fisher's exact test p value | — | *0.0256 |

CI, confidence interval;
PCP, Pneumocystis pneumonia;
*p < 0.05

### Pan-Fungal Peptide 2Immunization Reduces Pneumocystis Colonization

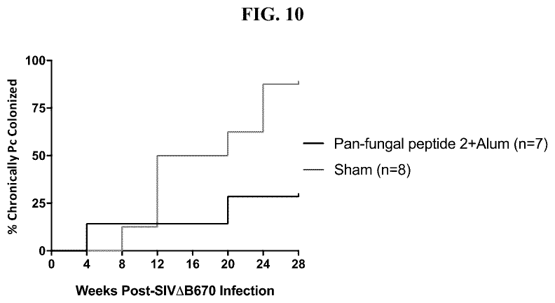

In addition to detection of Pneumocystis by first round PCR, BAL samples were assessed by nested PCR for colonization with Pneumocystis. FIG. 10 illustrates the time (in weeks) to chronic colonization (as determined by a positive nested PCR in two or more consecutive months) in both Pan-fungal peptide 2-immunized and sham-immunized cohorts. Chronic colonization was found to be significantly reduced in the Pan-fungal peptide 2-immunized cohort compared to the sham-immunized cohort by 28 weeks post infection (wpi), (*p 0.05).

Materials and Methods used in this Example are as follows:

### Animals

Fifteen (15) adult Chinese-origin Rhesus macaques (Macaca mulatta) were obtained from national primate centers or vendors and were housed in accordance with the NIH Guide for the Care and Use of Laboratory Animals in a BSL2+ primate facility.

### Vaccine Construction and Purification

A 90-amino recombinant protein Pan-fungal peptide 2 was cloned into the pET28b(+) expression vector (Novagen) in Escherichia coli BL21(DE3) pLysS (ThermoFisher, Scientific) and was purified by affinity chromatography. The recombinant Pan-fungal peptide 2 protein was used for immunization and enzyme-linked immunosorbent assay (ELISA).

### Immunization and Study Design

Seven macaques were intramuscularly immunized with 100 μg of recombinant Pan-fungal peptide 2 and aluminum hydroxide (Imject Alum, Thermo Scientific) mixed in a 1:1 ratio. A control cohort of eight sham-immunized (vaccinated) macaques received PBS and alum. Animals were rested for eight weeks, after which time Pan-fungal peptide 2-immunized animals were boosted with 50 μg of Pan-fungal peptide 2 and alum, and sham-immunized animals were sham inoculated as described above. 8 weeks following the boost, the NHP animals were infected intravenously with SIV/Delta B670 (1:100 in PBS), tissue culture infectious dose of 50% (TCID₅₀=2.6×10⁵). Viral infection was monitored at weekly time points for 4 weeks and monthly thereafter for up to 32 weeks after infection. Bronchoalveolar lavage (BAL) fluid was collected monthly to monitor for the presence of Pneumocystis infection.

### Pneumocystis Challenge and Determination of Pneumocystis Infection

Pneumocystis (Pc) cannot be reliably cultured in vitro. Thus, Pneumocystis challenge of Pan-fungal peptide 2-immunized and sham-immunized Rhesus macaques was performed via natural airborne transmission by co-housing these animals as described previously by Kling et al. (2010, Infect. Immun., 78(10):4320-4330). Pneumocystis pneumonia (PCP) was defined as detection of Pneumocystis in BAL fluid by first-round PCR and/or by microscopy-based detection of Pneumocystis in a BAL smear. Nested PCR was performed by amplifying 5 μL of the first-round product, as previously described (Savoia, D. et al., 1997, Diagn Microbiol Infect Dis, 29(2):61-65). Chronic pulmonary Pneumocystis colonization was defined as detection of Pneumocystis DNA in the nested round of PCR in two or more consecutive months. To control for the DNA quality in BAL fluid samples, PCR for detection of β-globin was also performed on BAL samples at each timepoint (method described by Croix, D A et al., 2002, AIDS Res Hum Retroviruses, 18(5):391-401).

### Statistical Analysis

All statistical analyses were performed using GraphPad Prism (GraphPad Software, La Jolla, CA). Fisher's exact test used to determine the association between Pan-fungal peptide 2immunization (vaccination) and the incidence of PCP in the vaccinated group compared with the sham-immunized group. Differences in PCP diagnosis between Pan-fungal peptide 2immunized and sham-immunized cohorts were compared using a Kaplan-Meier test. The relative risk of PCP was calculated using Koopman asymptotic score. Chronic Pc colonization was evaluated by Mantel-Cox test.

### OTHER EMBODIMENTS

From the foregoing description, it will be apparent that variations and modifications may be made to the aspects and embodiments described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunogenic peptide, or a polynucleotide encoding an immunogenic peptide, having at least 95% or more sequence identity to amino acid sequence PDDGKTMEGPDILVLRAFINGVQNGRDGKGSIYVFASGNGGGFEDNCNFDGYTNSIY SITVGAIDRKGLHPSYSEACSAQLVVTYSSGSG as set forth in SEQ ID NO: 2 (Pan-fungal peptide 2), or an immunogenic portion thereof.

2. An immunogenic peptide, or a polynucleotide encoding an immunogenic peptide, wherein the immunogenic peptide comprises or consists of amino acid sequence PDDGKTMEGPDILVLRAFINGVQNGRDGKGSIYVFASGNGGGFEDNCNFDGYTNSIY SITVGAIDRKGLHPSYSEACSAQLVVTYSSGSG as set forth in SEQ ID NO: 2 (Pan-fungal peptide 2), or an immunogenic portion thereof.

3. An immunogenic composition comprising an effective amount of the peptide of claim 1, or a polynucleotide encoding the peptide, and a pharmaceutically acceptable carrier, vehicle, or excipient.

4. An immunogenic composition comprising an effective amount of the peptide of claim 2, or a polynucleotide encoding the peptide, and a pharmaceutically acceptable carrier, vehicle, or excipient.

5. The immunogenic composition of claim 3, wherein the composition further comprises an adjuvant, optionally, wherein the adjuvant is alpha-galactosylceramide (αGC).

6. The immunogenic composition of claim 4, wherein the composition further comprises an adjuvant, optionally, wherein the adjuvant is alpha-galactosylceramide (αGC).

7. The immunogenic composition of claim 3, wherein the composition protects against disease or symptoms thereof caused by fungal infection.

8. The immunogenic composition of claim 4, wherein the composition protects against disease or symptoms thereof caused by fungal infection.

9. The immunogenic composition of claim 7, wherein the disease or the symptoms thereof are caused by infection by one or more of Aspergillus, Candida, Cryptococcus, or Pneumocystis fungal pathogens.

10. The immunogenic composition of claim 8, wherein the disease or the symptoms thereof are caused by infection by one or more of Aspergillus, Candida, Cryptococcus, or Pneumocystis fungal pathogens.

11. The immunogenic composition of claim 9, wherein the disease or the symptoms thereof are caused by infection by one or more of Aspergillus, Candida, or Cryptococcus fungal pathogens.

12. The immunogenic composition of claim 10, wherein the disease or the symptoms thereof are caused by infection by one or more of Aspergillus, Candida, or Cryptococcus fungal pathogens.

13. The immunogenic composition of claim 7, wherein the disease is selected from a pulmonary disease, a pulmonary dysfunction, Pneumocystis pneumonia (PCP), aspergillosis, invasive pulmonary aspergillosis (IPA), or a symptom thereof.

14. The immunogenic composition of claim 8, wherein the disease is selected from a pulmonary disease, a pulmonary dysfunction, Pneumocystis pneumonia (PCP), aspergillosis, invasive pulmonary aspergillosis (IPA), or a symptom thereof.

15. The immunogenic composition of claim 7, wherein the disease or a symptom thereof afflicts a human subject who is immunocompromised or immunosuppressed and/or is a pre-transplant subject or a post-transplant subject.

16. The immunogenic composition of claim 8, wherein the disease or a symptom thereof afflicts a human subject who is immunocompromised or immunosuppressed and/or is a pre-transplant subject or a post-transplant subject.

17. The immunogenic composition of claim 15, wherein the immunocompromised or immunosuppressed subject is being treated for cancer, an immunodeficiency disease, a congenital disease, an autoimmune disease, human immunodeficiency virus (HIV), rheumatoid arthritis, or psoriasis.

18. The immunogenic composition of claim 16, wherein the immunocompromised or immunosuppressed subject is being treated for cancer, an immunodeficiency disease, a congenital disease, an autoimmune disease, human immunodeficiency virus (HIV), rheumatoid arthritis, or psoriasis.

19. A vaccine comprising an effective amount of the immunogenic peptide of claim 1, a polynucleotide encoding the peptide, or a pharmaceutically acceptable composition thereof.

20. A vaccine comprising an effective amount of the immunogenic peptide of claim 2, a polynucleotide encoding the peptide, or a pharmaceutically acceptable composition thereof.
